(19) United States
(12) Patent Application Publication
(10) Pub. No.: US 2026/0365969 A1
(43) Pub. Date: Nov. 19, 2026

(54) ZWITTERIONIC HYDROGELS FOR THE CULTURE, EXPANSION, AND PROTECTION OF CELLS AND CELL-DERIVED PRODUCTS

(71) Applicant: CORNELL UNIVERSITY, Ithaca, NY (US)

(72) Inventors: Shaoyi JIANG, Ithaca, NY (US); Di LIU, Ithaca, NY (US); Erica WAGNER, Ithaca, NY (US)

(73) Assignee: CORNELL UNIVERSITY, Ithaca, NY (US)

(21) Appl. No.: 19/486,090

(22) PCT Filed: May 30, 2024
(86) PCT No.: PCT/US2024/031552

§ 371 Date: Nov. 20, 2025

Related U.S. Application Data
(60) Provisional application No. 63/469,660, filed on May 30, 2023.

(51) Int. Cl. C12N 5/00; A61K 9/00; A61K 35/545; A61K 40/11; A61K 40/15; A61L 27/18; A61L 27/22; A61L 27/36; A61L 27/52; A61L 27/54; C12N 5/074

(52) U.S. Cl. CPC C12N 5/0012; A61K 9/0024; A61K 35/545; A61K 40/11; A61K 40/15; A61L 27/18; A61L 27/22; A61L 27/3604; A61L 27/52; A61L 27/54; C12N 5/0696; C12N 2533/30; C12N 2537/10

(57) ABSTRACT

A hydrogel composition comprising a star polymer core containing at least three arms, wherein each arm contains a copolymer comprising zwitterionic monomer units and peptide-functionalized monomer units, wherein the peptide functions as a cell adhesive ligand, and wherein the hydrogel composition contains a biodegradable or non-biodegradable linker. Also described herein are methods of producing the hydrogel composition as well as methods of culturing and containing cells comprising combining cells with the hydrogel composition under conditions to promote cell survival, maintenance, cell product production, or cell growth.

### CROSS REFERENCE TO RELATED APPLICATIONS

This application claims the benefit of priority from U.S. Provisional Application No. 63/469,660, filed on May 30, 2023, which is herein incorporated by reference in its entirety.

### GOVERNMENT SUPPORT

This invention was made with government support under grant number DGE 2139899 awarded by the National Science Foundation. The government has certain rights in the invention.

### FIELD OF THE INVENTION

The present invention generally relates to zwitterionic hydrogel compositions and uses thereof for the culture, expansion, and protection of cells and cell-derived products, such as stem cells, stem cell-derived cells and organoids, spheroids, cancer organoids, healthy organoids, diseased organoids, pancreatic islets, neutrophils, monocytes, T cells, and NK cells. The present invention more specifically relates to zwitterionic hydrogel compositions that include a peptide-functionalized portion that functions as a cell adhesive portion and a biodegradable or non-biodegradable linker and uses thereof for the culture, expansion, and protection of cells and cell-derived products.

### BACKGROUND OF THE INVENTION

Hydrogels have long been of interest for biological and biomaterial applications due to their high water content that mimics the interstitial tissue environment, ensures high diffusive permeability, and provides biomimetic mechanical strengths. Particular interest has been given to PEG hydrogels and poly(2-hydroxyethyl methacrylate) (pHEMA) hydrogels because, in addition to the general properties of hydrogels, they are also commonly considered to be low fouling, bio-inert, and versatile. However, PEG and pHEMA hydrogels have substantial drawbacks, including lower than ideal hydration ability, higher than ideal susceptibility to fouling, difficulty functionalizing the hydroxy group, and susceptibility to oxidation.

Zwitterionic hydrogels have also been studied for biological applications. However, while zwitterionic hydrogels have mitigated some of the problems with PEG and pHEMA hydrogels, currently known zwitterionic hydrogels are substantially limited due to their cumbersome synthesis, poor hydrolytic stability, and non-ideal gelation rate, the latter of which results in heterogeneous gels in which cells are not fully and homogeneously dispersed.

### SUMMARY

The present invention solves the problems noted above with current zwitterionic hydrogels, particularly as applied to biological applications. The zwitterionic hydrogels of the present invention are substantially non-biodegradable, are highly resistant to hydrolysis, have low toxicity, can be easily dissolved to recover encapsulated cells, and have much more favorable gelation times, the latter of which results in homogeneous gels in which cells are fully and homogeneously dispersed.

In a first aspect, the present disclosure provides hydrogel compositions containing a star polymer core that contains at least three arms, wherein each arm contains a copolymer comprising zwitterionic monomer units and peptide-functionalized monomer units, wherein the peptide functions as a cell adhesive ligand, and wherein the hydrogel composition contains a biodegradable or non-biodegradable linker. The term “monomer units,” as used herein, is understood to mean monomer units that have undergone polymerization. The zwitterionic monomer units and peptide-functionalized monomer units are both in the copolymer. The copolymer may be any of the copolymers known in the art, such as block, random, alternating, periodic, and branched copolymers. In some embodiments, the star polymer core (or star polymer altogether) is hydrolytically stable (i.e., is not hydrolytically degradable), and the star polymer core may or may not be biodegradable or non-biodegradable. By being hydrolytically stable, the star polymer core (or star polymer altogether) is stable from hydrolysis over a period of at least 1, 2, 3, 4, or 5 days or even 1, 2, or 3 weeks (or within a range anywhere therein).

In particular embodiments, the hydrogel composition contains first (10) units and second (2^{nd}) units bonded to each other and has the following structure:

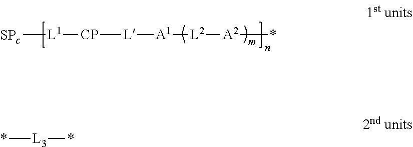

The variables in the above structures are defined as follows: SP_{c} is a star polymer core containing n arms, wherein n is at least 3; L¹ and L′ are independently selected from a bond or a linker containing at least one carbon atom and optionally one or more heteroatoms selected from O, N, and S; CP is a copolymer comprising zwitterionic monomer units and peptide-functionalized monomer units, wherein the peptide functions as a cell adhesive peptide (ligand); A¹ is a reaction product between first and second reactive functional groups; L², independently in each instance, is a linker containing at least one carbon atom and optionally one or more heteroatoms selected from O, N, and S; A², independently in each instance, is a reaction product between first and second reactive functional groups; m is typically 0, 1, 2, or 3; and L³ is a biodegradable or non-biodegradable linker; wherein the asterisks (*) in 1^{st} units represent connection points with asterisks in 2^{nd} units, and the hydrogel composition has a multiplicity of 1^{st} and 2^{nd} units. Notably, the zwitterionic moiety in the zwitterionic monomer units can be any kind of zwitterionic moiety, including, for example, poly(carboxybetaine), poly(2-methacryloyloxyethyl phosphorylcholine), poly(sulfobetaine) and trimethylamine N-oxide derived polymer (PTMAO). The zwitterionic moiety is not limited to any of the specific zwitterionic moieties disclosed herein unless otherwise specified. Similarly, the cell adhesive peptide can be any type of cell adhesive peptide, including, for example, RGD, RGDS, c(RGDyK), IKVAV, YIGSR, PHSRN, and REDV, DDFVFYVGGYPS, TALKFYLQGPEP, LRLVSYSGVLFF, ASKAIQVFLLGG, LDGTGFARISFD, and QQNLGSVNVSTG. The cell adhesive peptide is not limited to any of the specific cell adhesive peptides disclosed herein unless otherwise specified.

In another aspect, the present disclosure is directed to a method of forming the hydrogel composition described above. The method generally includes the following steps:

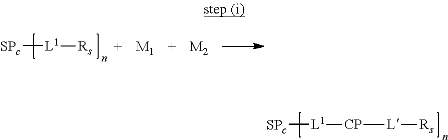

wherein:
SP_{c} is a star polymer (SP) core containing n arms, each arm containing a bond or linker (L¹) attached to a terminal reactive site (Rₛ) from which polymer growth can be initiated, wherein n is at least 3; wherein L¹ and L′ are independently selected from a bond or a linker containing at least one carbon atom and optionally one or more heteroatoms selected from O, N, and S;
M₁ is a zwitterionic monomer and M₂ is peptide-functionalized monomer, wherein the peptide functions as a cell adhesive ligand; and
CP is a copolymer containing: (a) zwitterionic monomer units resulting from polymerization of M₁ and (b) peptide monomer units resulting from polymerization of M₂;
step (ii): functionalizing SP_{c} [symbol] L¹-CP-L′-Rₛ]ₙ with a first reactive functional group R¹ to form the following structure: SP_{c} [symbol] L¹-CP-L′-R¹]ₙ, wherein steps (i) and (ii) are optionally performed simultaneously; and
step (iii) reacting SP_{c} [symbol] L¹-CP-L′-R¹]ₙ with R²-L³-R³ to form the hydrogel composition containing 1^{st} and 2^{nd} units, wherein:
R² and R³ are second and third reactive functional groups, respectively, and L³ is a biodegradable or non-biodegradable linker; wherein R¹ and R² react to form a reaction product A¹, and R¹ and R³ react to form another reaction product A¹, to result in the 1^{st} units and 2_{nd} units having the following structures:

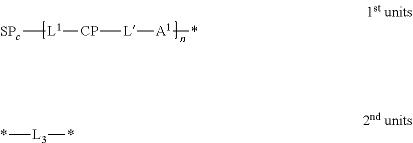

wherein the asterisks (*) in 1^{st} units represent connection points with asterisks in 2^{nd} units, and the hydrogel composition has a multiplicity of 1^{st} and 2^{nd} units.

In some embodiments, step (ii) further comprises an intermediate crosslinking step as follows:
reacting SP_{c} [symbol] L¹-CP-L′-R¹]ₙ with R^{1′}-L²-R^{2′} to form the following structure: SP_{c} [symbol] L¹-CP-L′-A¹-L²-R^{2′}]ₙ,
   wherein R¹, R^{1′}, and R^{2′} are reactive functional groups, and R¹ and R^{1′} react to form a reaction product A¹, and L² is a linker containing at least one carbon atom and optionally one or more heteroatoms selected from O, N, and S; and
reacting SP_{c} [symbol] L¹-CP-L′-A¹-L²-R^{2′}]ₙ with R²-L³-R³ to form the hydrogel composition containing 1^{st} and 2^{nd} units, wherein:
   R² and R³ are second and third reactive functional groups, respectively, and L³ is a biodegradable or non-biodegradable linker; wherein R^{2′} and R² react to form a reaction product A², and R^{2′} and R³ react to form another reaction product A², to result in the 1^{st} units and 2^{nd} units having the following structures:

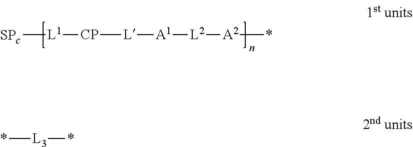

wherein the asterisks (*) in 1^{st} units represent connection points with asterisks in 2^{nd} units, and the hydrogel composition has a multiplicity of 1^{st} and 2^{nd} units.

In another aspect, the present disclosure is directed to a method of culturing and containing cells comprising combining cells with any of the hydrogel compositions described above under conditions to promote cell survival, maintenance, cell product production, or cell growth. The cell can be any type of cell, including, for example, stem cells, stem cell-derived cells and organoids, spheroids, cancer organoids, healthy organoids, diseased organoids, pancreatic islets, neutrophils, monocytes, T cells, and NK cells. The cell is not limited to any of the specific cell types disclosed herein unless otherwise specified.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Scheme summarizing the challenges associated with the synthesis of key PCB gel components and hydrogel formation in previous studies and the solutions developed in this work.

FIG. 2. Scheme showing improved synthesis of various linear and 4-arm poly(zwitterionic acrylamide)s and the structures of monomers (CBAA-1, CBAA-2, SBAA-3, NOAA), initiators (4f-PETP, 4f-PAMAM, HEBiB), and ligand (Me₆Tren) used in this work. Method A is well suited when a water-soluble initiator such as 4f-PAMAM and HEBiB are used while method B is more suitable when a hydrophobic initiator is used.

FIGS. 3a-3e. FIG. 3a is a graph showing degradation of PETP-PCB (28 kDa, prepared using method B) at physiological pH (PBS, 37 °C, 6 days) and in strong alkaline solution (0.1 M NaOH, RT, 1 h). FIG. 3b is a graph showing degradation of PAMAM-PCB (133 kDa, prepared using method A) at physiological pH (PBS, 37 °C, 6 days) and in strong alkaline solution (0.1 M NaOH, RT, 1 h). FIG. 3c is a bar chart showing Young's modulus and equilibrium mass swelling ratio of PCB hydrogels prepared from different compositions and molecular weights, wherein N₃—(PEG)₃-N₃ was used as the crosslinker, (N₃:DBCO=1:1), N=4 samples for each composition; Mean±SD. FIG. 3d shows]H NMR spectra of 60 kDa 4-arm end-functionalized PCB, with PCB-N₃ (top), PCB-NH₂ (middle), PCB-DBCO (bottom); left insets (7-8.2 ppm) display the resonance corresponding to the chain-end functionality, right insets (0.9-1.3 ppm) display the resonance of the α-end (methyl) of the polymer. FIG. 3e shows GPC chromatograms of 60 kDa 4-arm end-functionalized PCB, with PCB-N₃ (top), PCB-NH₂ (middle), PCB-DBCO (bottom).

FIG. 4. Scheme showing post-polymerization modification of 4-arm azide-terminated PCB (PCB-N₃). Star-shaped end-functionalized PCB can be synthesized quantitatively by reacting an alkyne-containing bifunctional molecule with PCB-N₃ or through an amide coupling reaction of PCB-NH₂ with a bifunctional NHS ester.

FIGS. 5a-5f. FIG. 5a shows representative bright field imaging of iPSC cell growth in PCB hydrogels over 7 days of culture. FIG. 5b shows live (green)/dead (red) staining of iPSCs after encapsulation and expansion in the PCB hydrogel at days 1, 3, 5, and 7. FIG. 5c is a chart showing viability quantifications of live/dead staining. N=4 gels per timepoint; Mean±SEM. FIG. 5d is a chart showing fold expansion from day 0 of iPSCs expanded in the PCB hydrogel over 7 days of culture. N=4 gels per timepoint; Mean±SEM. FIG. 5e is a chart showing total cells per mL gel harvested from the PCB hydrogel after 5 and 7 days of culture. N=4 gels per timepoint; Mean±SEM. FIG. 5f is a karyotype analysis of iPSCs expanded in the PCB hydrogel over 7 days.

FIGS. 6a-6d. FIG. 6a shows immunofluorescence staining of pluripotency markers OCT4, NANOG, SSEA-3, and SSEA-4 in iPSC spheroids cultured for 5 days in the PCB hydrogel. FIG. 6b is a graph showing RT-qPCR results of NANOG and OCT4 gene expression levels in iPSCs expanded on Matrigel® compared to inside the PCB hydrogel after 5 days of culture. N=4 samples for each group; Mean±SEM. *p<0.05; **p<0.01; ***p<0.001 by Student's t-test. FIG. 6c is a representative image of AP staining of PCB-cultured iPSCs. FIG. 6d is a graph showing quantified % of AP+ spheroids generated from the PCB hydrogel after 5 and 7 days of culture. N=4 samples for each timepoint; Mean±SEM.

FIGS. 7a-7d. FIG. 7a shows immunofluorescence staining of germ layer specific markers in iPSCs expanded in the PCB hydrogel and then differentiated to ectoderm, mesoderm, and endoderm lineages. FIG. 7b presents graphs showing RT-qPCR results of germ layer markers for PCB-derived iPSCs differentiated to the ectoderm, mesoderm, and endoderm germ layers compared to undifferentiated iPSCs. N=4 samples for each group; Mean±SEM. *p<0.05; **p<0.01; ***p<0.001; ****p<0.0001; ns=no significance by Student's t-test. FIG. 7c shows immunofluorescence staining of germ layer specific markers in iPSCs differentiated inside the PCB hydrogel. FIG. 7d presents graphs of RT-qPCR results of germ layer markers in iPSCs differentiated inside the PCB hydrogel to the ectoderm, mesoderm, and endoderm germ layers compared to undifferentiated iPSCs. N=4 samples for each group; Mean±SEM. *p<0.05; **p<0.01; ***p<0.001; ****p<0.0001 by Student's t-test.

FIG. 8. Scheme showing an exemplary synthesis of a representative functionalized copolymer of carboxybetaine acrylamide monomer (CBAA) and N-acryl peptides that serve to promote cell adhesion.

### DETAILED DESCRIPTION

In a first aspect, the present disclosure is directed to hydrogel compositions useful for culturing and containing cells. The hydrogel compositions contain a star polymer core (SP_{c}) that contains at least three arms, wherein each arm contains a copolymer that contains zwitterionic monomer units and peptide-functionalized monomer units. The peptide functions as a cell adhesive ligand. The hydrogel compositions also contain a biodegradable or non-biodegradable linker.

The term “monomer units,” as used herein, is understood to mean monomer units that have undergone polymerization. The zwitterionic monomer units and peptide-functionalized monomer units are both in the copolymer. The copolymer may be any of the types of copolymers known in the art, such as block, random, alternating, periodic, and branched copolymers. In some embodiments, the star polymer core (or star polymer altogether) is hydrolytically stable (i.e., is not hydrolytically degradable), and the star polymer core may or may not be biodegradable or non-biodegradable. By being hydrolytically stable, the star polymer core (or star polymer altogether) is stable from hydrolysis over a period of at least 1, 2, 3, 4, or 5 days or even 1, 2, or 3 weeks (or within a range anywhere therein).

In particular embodiments, the hydrogel composition contains first (1^{st}) units and second (2^{nd}) units bonded to each other and has the following structure:

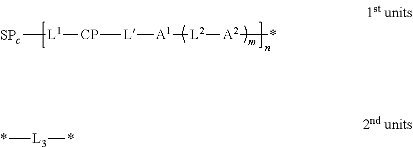

The variable SP_{c} is a star polymer core containing n arms, wherein n is at least 3. The variable n may be, for example, precisely or at least 3, 4, 5, or 6. The SP_{c} is typically a dendrimer. The SP_{c} may be, for example, a poly(amidoamine) (PAMAM) dendrimer, poly(propyleneimine) (PPI) dendrimer or poly(etherhydroxylamine) (PEHAM) dendrimer, wherein the dendrimer generation is typically 0-3, ore more particularly 0, 1, 2, or 3. In particular embodiments, the SP_{c} is a polyamidoamine (PAMAM) core. Such dendrimers are well known in the art, as evidenced by, for example, S. Svenson, Eur. J. Pharm. Biopharm., 71(3):445-462, 2009, the contents of which are herein incorporated by reference. The number of arms (n) may, in some embodiments, be precisely or at least 3, 4, 5, or 6.

The term “star polymer core” (i.e., SP_{c}), as used herein, refers to a portion of the star polymer from which the arms of the star polymer emanate; thus, the star polymer core refers to a central portion of the polymer that does not include the arms although it includes bonds to the arms. In the case of a PAMAM dendrimer containing four arms, the star polymer core (SP_{c}) may have an ethylenediamine (EDA) structure, i.e., N—CH₂CH₂—N, in which each N contains two single covalent bonds (not shown), with each bond connecting to an arm, thereby arriving at four arms. Similarly, in the case of a poly(propyleneimine) (PPI) dendrimer containing four arms, the star polymer core (SP_{c}) may have a tetramethylene diamine structure, i.e., N—CH₂CH₂CH₂CH₂—N, in which each N contains two single covalent bonds (not shown), with each bond connecting to an arm, thereby arriving at four arms. Similarly, in the case of a poly(etherhydroxylamine) (PEHAM) dendrimer (or polyetheramine dendrimer), the star polymer core (SP_{c}) may have the structure N—(CH₂CH₂O)ᵥCH₂CH₂—N wherein v is precisely or at least 1 or 2, in which each N contains two single covalent bonds (not shown), with each bond connecting to an arm, thereby arriving at four arms. An example of a three-arm star polymer are those within the class of poly(alkyl aryl ether) dendrimers containing a phloroglucinolyl (1,3,5-trioxylphenyl) core, such as described in J. Nithyanandhan et al., J. Org. Chem., 67, 17, 6282-6285, 2002, the contents of which are incorporated herein by reference. An example of an eight-arm star polymer are those synthesized from a tripentaerythritol core, as described in B. Chen et al., Bioconjugate. Chem., 22, 4, 617-624, 2011, the contents of which are herein incorporated by reference.

More generally, the star polymer core may have the formula N—(CH₂CH₂O)ᵥ(CH₂)ᵣ—N, wherein v is 0, 1, 2, or a higher number and r is typically 1-6, and wherein, optionally, any one or more H atoms in CH₂ groups may be substituted with one or more methyl groups to result in a branched star polymer core. Typically, each arm contains two or more branches depending on the generation number of the star polymer. The star polymer may have any number of arms and any number of branches on each arm. For example, the star polymer may be a zero, first, second, third, or higher generation star polymer (or more specifically, dendrimer).

The variables L′ and L′ are independently selected from a bond or a linker containing at least one, two, three or more carbon atoms and optionally one or more heteroatoms, such as one or more selected from O, N, and S. The linker L′ typically corresponds to a portion of an arm of the star polymer connecting the core (SP_{c}) with the copolymer portion (CP). In the case of a PAMAM dendrimer, L′ typically is or includes the following type of linkage: [(CH₂)ᵣC(O)NH]ₘ, wherein r is typically an integer within a range of 1-6 (e.g., 2), and m is typically 2, 3, or more. However, L¹ in the PAMAM dendrimer or other type of star polymer may have any of a variety of different types of structures, such as, for example, —(CH₂)ᵣ, wherein r is typically 1-6; —(CH₂CH₂O)ᵥ—CH₂CH₂—NH—, where v is precisely or at least 1 or 2; and [—(CH₂CH₂O)ᵥ(CH₂)ᵣC(O)NH]ₘ.

The linker may be linear or branched. In the case of a branched linker, the linker (e.g., L¹) may have any number of branching points connected to further linking portions (e.g., L^{1a}, L^{1b}, L^{1c}, etc), wherein the further linking portions in branched portions are all considered to be within the scope of L¹. Thus, the linker includes the linking portions in arms of first, second, third, or higher dendrimers connecting to the CP portion. Notably, in the case where L¹ is linear with no branching portions, one CP portion will be contained per each L¹ and each arm of the star polymer. However, in the case where L¹ contains branching portions, a CP-L′-A¹-(L²-A²)ₘ- portion, as shown in the formula of 1^{st} units, will emanate from and thus be present in each branch of L¹, which thus results in more than one CP-L′-A¹-(L²-A²)ₘ- portion present in each arm of n arms. The formula shown above for 1^{st} units is intended to encompass both linear and branching scenarios for L¹, and thus, the possibility of having 1, 2, 3, 4 or more CP-L′-A¹-(L²-A²)ₘ- portions in each arm of the star polymer. The linker L′ is independently selected from a bond or any type of linker such as any of the exemplary linkers provided above for L¹. L², independently in each instance, is a linker containing at least one carbon atom and optionally one or more heteroatoms selected from O, N, and S. The linker L² may be any of the exemplary linkers provided above for L¹. Moreover, any of the star polymer cores, as described earlier above, can be combined with any of the above described L¹, L′, and L² linkers.

The variable CP is a copolymer containing zwitterionic monomer units and peptide-functionalized monomer units, wherein the peptide functions as a cell adhesive peptide (ligand). The term “monomer units,” as used herein, is understood to mean monomer units that have undergone polymerization. The zwitterionic monomer units and peptide-functionalized monomer units are both in the copolymer. In some embodiments, zwitterionic monomers are electronically neutral monomers that include equal numbers of positive and negative charges (e.g., one of each). However, as further discussed below, the zwitterionic monomers, may, in some embodiments, be a combination of positively-charged monomers and negatively charged monomers.

The copolymer (CP) may be any of the types of copolymers known in the art, such as block, random, alternating, periodic, and branched copolymers. In specific embodiments, the CP is a block copolymer containing precisely or at least one block of the zwitterionic polymer and precisely or at least one block of the peptide-functionalized polymer. In other specific embodiments, the CP is a random copolymer containing zwitterionic units and peptide-functionalized units randomly present in the copolymer.

The zwitterionic moiety in the zwitterionic monomer units can be any kind of zwitterionic moiety, including, for example, poly(carboxybetaine), poly(2-methacryloyloxyethyl phosphorylcholine), poly(sulfobetaine) and trimethylamine N-oxide derived polymer (PTMAO). The zwitterionic moiety is not limited to any of the specific zwitterionic moieties disclosed herein unless otherwise specified.

As used herein, the term “zwitterionic polymer” includes polymers prepared by polymerizing a polymerizable zwitterionic monomer, which results in a zwitterionic polymer having 100 mole percent zwitterionic moieties (i.e., each repeating unit of the zwitterionic polymer is a zwitterionic moiety); or the term “zwitterionic polymer” refers to a copolymer prepared by copolymerizing a polymerizable zwitterionic monomer and a polymerizable comonomer, which provides a zwitterionic copolymer containing less than 100 mole percent zwitterionic moieties (e.g., when the polymerizable zwitterionic monomer and the polymerizable comonomer are present in equal proportions in the polymerization mixture, the product is a zwitterionic polymer having 50 mole percent zwitterionic moieties). In some embodiments, the zwitterionic polymer may alternatively be prepared from monomers that can be converted to zwitterionic monomers, i.e., precursors of zwitterionic monomers. In some embodiments, the zwitterionic polymer contains a plurality of repeating units, each repeating unit comprising one positive and one negative charged moiety. The zwitterionic polymer typically contains at least or greater than 2, 5, or 10, and up to or less than 100, 200, 300, 400, 500, or 1000 units.

In some embodiments, CP contains a zwitterionic polymer block and may be a homopolymer or copolymer. The zwitterionic polymer block may have the following structure:

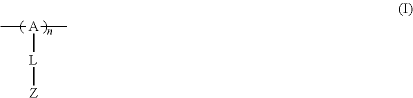

wherein A is a backbone of the polymer, L is a linking portion, Z is a zwitterionic moiety, and n is typically an integer of at least 2, 5, or 10. The backbone (A) may be, for example, polyester, polyether, polyurethane, polyamide, or polyhydrocarbon (e.g., polyethylene or polypropylene) backbone, and Z is a zwitterionic moiety. In particular embodiments, the backbone (A) may have any of the following structures:

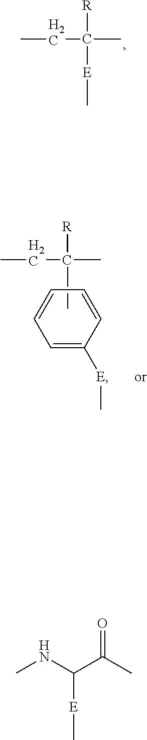

wherein R is selected from the group consisting of hydrogen and substituted or unsubstituted alkyl; and E is selected from the group consisting of substituted or unsubstituted alkylene, —(CH₂)ₚC(O)O—, and —(CH₂)ₚC(O)NR²—; p is typically an integer from 0 to 12; R² is selected from hydrogen and substituted or unsubstituted alkyl; and L is a straight or branched alkylene group optionally including one or more oxygen atoms. The subscript x is typically at least or greater than 2, 5, or 10, and up to or less than 100, 200, 300, 400, 500 or 1000 units.

In some embodiments, Z is selected from any of the following structures:

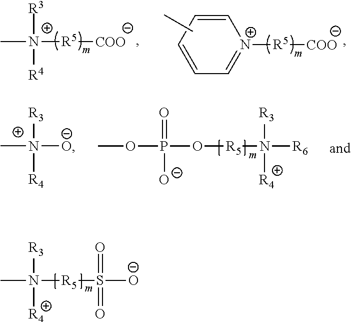

wherein R³, R⁴, and R⁶ are independently selected from the group consisting of hydrogen and substituted or unsubstituted alkyl group, R⁵ or R₅ is selected from the group consisting of substituted or unsubstituted alkylene, phenylene, and polyether groups, and m is an integer from 1 to 7; and x is an integer from 2 to 500.

In some embodiments, the zwitterionic polymer block has the following structure:

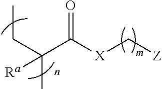

wherein: R^{a} is H or an alkyl group containing 1-3 carbon atoms; X is O or NR^{b}, wherein R^{b} is H or an alkyl group containing 1-3 carbon atoms; Z is a zwitterionic moiety; n is typically an integer of at least 2, 5, or 10; and m is an integer of at least 1. The group Z may be selected from, for example, carboxybetaine, sulfobetaine, phosphobetaine, and trialkylamine-N-oxide zwitterionic moieties.

In a first particular set of embodiments, Z contains a positively charged group directly bound to a negatively charged group. The zwitterionic polymer containing a positively charged group directly bound to a negatively charged group may have the following structure:

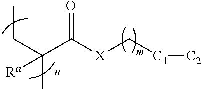

wherein R^{a}, X, n, and m are as defined above; and C₁ and C₂ are independently selected as positively charged and negatively charged groups to form a zwitterionic moiety C₁-C₂. The C₁-C₂ moiety may be, for example, a trialkylamine-N-oxide zwitterionic moiety.

When the C₁-C₂ moiety is a trialkylamine-N-oxide zwitterionic moiety, Formula (1a) can be further specified as the following formula:

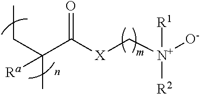

wherein R^{a}, X, n, and m are as defined above; and R¹ and R² are independently selected from R^{a}, as defined above. A particular example of a zwitterionic polymer of Formula (1a-1) is poly(trimethylamine N-oxide) polymer, i.e., pTMAO polymer, which may more specifically refer to poly(methacrylamidopropyl-trimethylamine N-oxide) (when X═N) or poly(methacryloylpropyl-trimethylamine N-oxide) (when X═O).

In a second particular set of embodiments, Z contains a positively charged group indirectly bound to a negatively charged group via a linker. The at least one zwitterionic polymer containing a positively charged group indirectly bound to a negatively charged group may have the following structure:

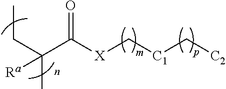

wherein R^{a}, X, n, and m are as defined above; C₁ and C₂ are independently selected as positively charged and negatively charged groups to form a zwitterionic moiety; and p is an integer of at least 1.

In some embodiments of Formula (1b), C₁ is an ammonium group, in which case Formula (1b) can be further specified as the following formula:

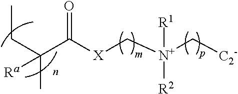

wherein R^{a}, X, n, m, and p are as defined above; C₂⁻ is a negatively charged group; and R¹ and R² are independently selected from R^{a}, as defined above. The group C₂⁻ may be, for example, a sulfonate or carboxylate group. Some examples of zwitterionic polymers of Formula (1b-1) when C₂ is sulfonate include poly(sulfobetaine)acrylamide, poly(sulfobetaine)acrylate, and poly(sulfobetaine)methacrylate. Some examples of zwitterionic polymers of Formula (1b-1) when C₂⁻ is carboxylate include poly(carboxybetaine)acrylamide, poly(carboxybetaine)acrylate, and poly(carboxybetaine)methacrylate.

When C₂ in Formula (1b-1) is a sulfonate group, Formula (1b-1) can be further specified as the following formula:

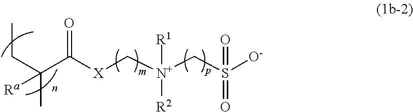

When C₂⁻ in Formula (1b-1) is a carboxylate group, Formula (1b-1) can be further specified as the following formula:

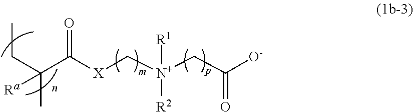

In other embodiments of Formula (1b), C₁ is a phosphonium group, in which case Formula (1b) can be further specified as the following formula:

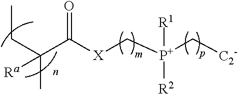

wherein R^{a}, X, n, m, and p are as defined above; C₂ is a negatively charged group; and R¹ and R² are independently selected from R^{a}, as defined above. The group C₂⁻ may be, for example, a sulfonate or carboxylate group. Some examples of zwitterionic polymers of Formula (1b-4) when C₂⁻ is sulfonate include poly(sulfophosphobetaine)acrylamide, poly(sulfophosphobetaine)acrylate, and poly(sulfophosphobetaine)methacrylate. Some examples of zwitterionic polymers of Formula (1b-1) when C₂⁻ is carboxylate include poly(carboxyphosphobetaine)acrylamide, poly(carboxyphosphobetaine)acrylate, and poly(carboxyphosphobetaine)methacrylate.

When C₂⁻ in Formula (1b-4) is a sulfonate group, Formula (1b-4) can be further specified as the following formula:

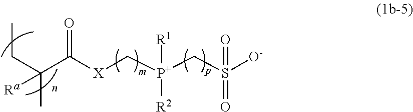

When C₂⁻ in Formula (1b-4) is a carboxylate group, Formula (1b-4) can be further specified as the following formula:

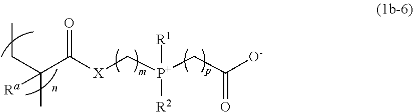

In other embodiments of Formula (1b), C₁ is a phosphate group, in which case Formula (1b) can be further specified as the following formula:

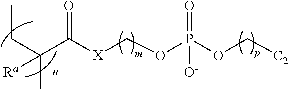

wherein R^{a}, X, n, m, and p are as defined in claim 4; and C₂⁺ is a positively charged group.

In some embodiments of Formula (1b-7), C₂⁺ is an ammonium group, in which case Formula (1b-7) can be further specified as the following formula:

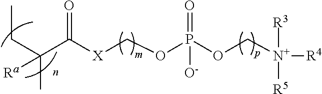

wherein R^{a}, X, n, m, and p are as defined above; and R³, R⁴, and R⁵ are independently selected from R^{a}, as defined above.

In some embodiments, the zwitterionic polymer is a betaine polymer. In other embodiments, the zwitterionic polymer is a poly(phosphatidylcholine) polymer, poly(trimethylamine N-oxide) polymer, poly(zwitterionic phosphatidylserine) polymer, or glutamic acid-lysine (EK)-containing polypeptide. In some embodiments, zwitterionic phosphatidyl serine comprises one neighboring positive charged moiety to balance the negative charge of the phosphoserine. In some embodiments, zwitterionic phosphatidyl serine comprises a compound as described in “De novo design of functional zwitterionic biomimetic material for immunomodulation” Science Advances, 29 May 2020, Vol. 6, Issue 22, (DOI: 10.1126/sciadv.aba0754) which is hereby incorporated by reference in its entirety.

Some examples of betaine polymers include poly(carboxybetaine), poly(sulfobetaine), and poly(phosphobetaine) polymers. Suitable poly(carboxybetaine)s can be prepared from one or more monomers selected from, for example, carboxybetaine acrylates, carboxybetaine acrylamides, carboxybetaine vinyl compounds, carboxybetaine epoxides, and mixtures thereof. In one embodiment, the monomer is carboxybetaine methacrylate. Representative monomers for making carboxybetaine polymers useful in the invention include carboxybetaine methacrylates, such as 2-carboxy-N,N-dimethyl-N-(2′-methacryloyloxyethyl) ethanaminium inner salt; carboxybetaine acrylates; carboxybetaine acrylamides; carboxybetaine vinyl compounds; carboxybetaine epoxides; and other carboxybetaine compounds with hydroxyl, isocyanates, amino, or carboxylic acid groups.

In a particular embodiment, the polymer is a poly(carboxybetaine methacrylate) (poly(CBMA)). In another particular embodiment, the polymer is a poly(carboxybetaine acrylamide) (poly(CBAA)).

In another embodiment, the zwitterionic polymer is a homopolymer that has a positive charge in the polymer backbone and a pendant carboxylic acid group and has the formula:

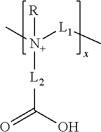

wherein R is selected from the group consisting of hydrogen and substituted or unsubstituted alkyl; L₁ and L₂ are independently a straight or branched alkylene group optionally including one or more oxygen atoms; and x is an integer from 2 to 500.

The term “zwitterionic polymer” also refers to a polymer having a substantially equal number of negative (anionic) charges and positive (cationic) charges that is prepared by copolymerizing a polymerizable negatively charged monomer and a polymerizable positively charged monomer, each present in substantially equal proportions in the polymerization mixture. The product of such a copolymerization is a zwitterionic polymer having 100 mole percent zwitterionic moieties, where each zwitterionic moiety is defined as a pair of repeating units: a repeating unit having a negative charge and a repeating unit having a positive charge. Such zwitterionic polymers are referred to as mixed charge copolymers. The term “zwitterionic polymer” also refers to a polymer prepared by copolymerizing a polymerizable negatively charged monomer, a polymerizable positively charged monomer, each present in substantially equal proportions in the polymerization mixture, and a polymerizable comonomer, which provides a zwitterionic polymer having less than 100 mole percent zwitterionic moieties (e.g., when the combination of polymerizable negatively charged monomer and polymerizable positively charged monomer and the polymerizable comonomer are present in equal proportions in the polymerization mixture).

In some embodiments, the mixed charge zwitterionic polymer has the following general formula:

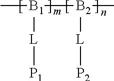

wherein B₁ and B₂ are polymer backbones and may, in some embodiments, be independently selected from X₁, X₂, and X₃, as described earlier above; R is selected from hydrogen and substituted or unsubstituted alkyl; E is selected from substituted or unsubstituted alkylene, —(CH₂)ₚC(O)O—, and —(CH₂)ₚC(O)NR²—, wherein p is an integer from 0 to 12; R² is selected from hydrogen and substituted or unsubstituted alkyl; L is a straight or branched alkylene group optionally including one or more oxygen atoms; P₁ is a positively charged group; P₂ is a negatively charged group, such as a carboxylic acid group; m is an integer from 1 to 500; and n is an integer from 1 to 500. In some embodiments, P₁ is nitrogen in an aromatic ring or NR₅R₆, wherein R₅ and R₆ are independently substituted or unsubstituted alkyl group.

The positively charged unit (P₁ containing unit) of the mixed-charge zwitterionic polymer can be derived from a monomer having a positively charged pendant group.

Representative monomers that can be used to derive the positively charged unit in the polymers of the present invention include 2-(dimethylamino)ethyl methacrylate, 2-(diethylamino)ethyl methacrylate, [2-(methacryloyloxy)ethyl]trimethylammonium chloride, and N-acetylglucosamine.

In one embodiment, the negatively charged unit of the mixed-charge zwitterionic polymer is derived from 2-carboxyethyl acrylate (CA), and the positively charged unit is derived from 2-(dimethylamino)ethyl methacrylate (DM). In another embodiment, the negatively charged unit is derived from 2-carboxyethyl acrylate (CA), and the positively charged unit is derived from 2-(diethylamino)ethyl methacrylate (DE). In another embodiment, the negatively charged unit is derived from 2-carboxyethyl acrylate (CA), and the positively charged unit is derived from [2-(methacryloyloxy)ethyl]trimethylammonium chloride (TM). In another embodiment, the negatively charged unit is derived from 2-carboxyethyl acrylate (CA), and the positively charged unit is derived from 2-aminoethyl methacrylate hydrochloride (NH-2). In some embodiments, the zwitterionic polymer excludes a polyalkylene oxide (polyalkylene glycol) segment, or the zwitterionic polymer more specifically excludes a polyethylene oxide or polypropylene oxide segment.

As noted earlier above, the variable CP is a copolymer of the zwitterionic polymer, as described above, and a cell adhesive peptide (ligand). The cell adhesive peptide can be any type of cell adhesive peptide, including, for example, RGD, RGDS, c(RGDyK), IKVAV, YIGSR, PHSRN, and REDV, DDFVFYVGGYPS, TALKFYLQGPEP, LRLVSYSGVLFF, ASKAIQVFLLGG, LDGTGFARISFD, CGRGDSG, and QQNLGSVNVSTG. The cell adhesive peptide is not limited to any of the specific cell adhesive peptides disclosed herein unless otherwise specified.

Moreover, any of the star polymer cores, as described earlier above, can be combined with any of the above described L¹ and L′ linkers and any of the above described copolymer (CP) moieties, including any of the zwitterionic units (or blocks) and any of the peptide-functionalized monomer units (or blocks). The star polymer may be a zero, first, second, third, or higher generation dendrimer in combination with any specific selection of SP_{c}, L¹, L′, and CP moieties.

A¹ and A² are, independently, reaction products between first and second reactive functional groups. A¹ and A² are thus linkages arising from the reaction between first and second reactive functional groups. A very large number of possible reactive groups are known in the art, and any of these may be appropriately paired and reacted for the purpose of the present invention to result in a reaction product A¹ and A². In some embodiments, A¹ and/or A² is a reaction product between any of the following pairs of reactive functional groups: (i) an azide and a terminal alkyne; (ii) an azide and a cycloalkyne; (iii) a thiol and a maleimide; (iv) a thiol and a vinylsulfone; (v) a thiol and a pyridyl disulfide; (vi) a thiol and an arylate; or (vii) an amine and a succinimidyl ester. In particular embodiments, A¹ and/or A² is a reaction product between an azide and a cycloalkyne. The cycloalkyne may be, for example, azadibenzocyclooctyne (DBCO), bicyclo[6.1.0]nonyne (BCN), or a difluorinated cyclooctyne (DIFO).

The variable m is typically 0, 1, 2, or 3. When m is 0, the optional additional linkage denoted as L²-A² is not present. When m is 1, a single instance of the optional additional linkage denoted as L²-A² is present. When m is 2, two instances of the optional additional linkage denoted as L²-A² are present and connected to each other, i.e., as L²-A²-(L²-A²)-, wherein each instance of L² and A² is independently selected. When m is 3, three instances of the optional additional linkage denoted as L²-A² are present and connected to each other, i.e., as L²-A²-(L²-A²)-(L²-A²)-, wherein each instance of L² and A² is independently selected.

The above variables (SP_{c}, L¹, CP, L′, A¹, L², A², m and n) are all located in first (1^{st}) units of the hydrogel composition. In the hydrogel composition, the above described first units are crosslinked with -L³- in second (2^{nd}) units, wherein L³ is a biodegradable or non-biodegradable linker. More specifically, the asterisks (*) in 1^{st} units represent connection points (i.e., covalent bonds) with asterisks in 2^{nd} units, and the hydrogel composition has a multiplicity of 1^{st} and 2^{nd} units. The multiplicity of units may be, for example, precisely or at least or greater than 10, 20, 50, 100, 1000, or more likely, many thousands, of 1^{st} and 2^{nd} units interconnected with each other. In a first set of embodiments, L³ is biodegradable linker, such as a matrix metalloproteinase (MMP) degradable peptide, as well known in the art (e.g., GKKCGPQGIWGQCKKG, as described in Y. Li et al., Biomaterials, 268: 120535, January 2021, which is herein incorporated by reference). Some other types of biodegradable linkers include ester-containing oligomers, such as PLA- or PGA-containing oligomers. In a first set of embodiments, L³ is a non-biodegradable linker, such as a PEG linker. Some other types of non-biodegradable linkers include azide-terminated vinyl polymers, such as poly(carboxybetaine acrylamide).

In another aspect, the present disclosure is directed to a method of forming any of the hydrogel compositions described above. The method generally includes the following steps:

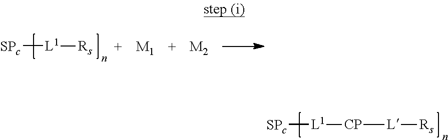

In step (i) above, the variables SP_{c}, L¹, L′, CP, and n are all as defined earlier above under the description of 1^{st} units. All definitions and possible combinations of these variables apply and can be made in the above step (i).

The variable Rₛ is a terminal reactive site (Rₛ) attached to the linker L¹. The terminal reactive site (Rₛ) is carbon atom attached to a reactive functional group. Polymer growth is initiated and propagated at the carbon atom of Rₛ. Thus, the polymer chain grows from the carbon atom of Rₛ with Rₛ retained at the terminal end of the polymer when polymer growth has ended. Functional groups that can serve as terminal reactive sites are well known in the art. Typically, the terminal reactive site (Rₛ) is an alkyl bromide, alkyl chloride, or alkyl iodide. However, other types of terminal reactive sites may be used, as well known in the art. In some embodiments, the terminal reactive site may be one appropriate for engaging in an atom transfer radical polymerization (ATRP) reaction. Some of the various conditions employed for ATRP reactions are described in, for example, K. Matyjaszewski et al., Chem. Rev., 101, 9, 2921-2990, 2001, the contents of which are herein incorporated by reference. In other embodiments, the terminal reactive site may be one appropriate for engaging in a reversible addition-fragmentation chain transfer (RAFT) polymerization reaction, typically a thiocarbonylthio end-group. Some of the various conditions employed for RAFT polymerization are described in, for example, M. Semsarilar et al., Macromol. Chem. Phys., 222(1), 1-30, 2000311, January 2021, the contents of which are herein incorporated by reference.

The variable M₁ in step (i) above is a zwitterionic monomer, which may be any of the zwitterionic monomers described in detail earlier above. The variable M₂ in step (i) above is a peptide-functionalized monomer, wherein the peptide functions as a cell adhesive ligand and may be any of the peptide-functionalized monomers described in detail earlier above. M¹ and M² are simultaneously or sequentially polymerized from the terminal reactive site (Rₛ) to form the copolymer CP, wherein CP is a copolymer resulting from the copolymerization of M¹ and M². In some embodiments, the copolymerization of M¹ and M² is simultaneous, thus resulting in a random copolymer CP. In other embodiments, the copolymerization of M¹ and M² is sequential, which results in a block copolymer containing a zwitterionic-containing block resulting from polymerization of M₁ and a peptide-containing block resulting from polymerization of peptide monomer units resulting from polymerization of M₂. Other means for incorporating CP into the star polymer are possible. For example, a precursor monomer of M¹ and/or M² may be polymerized followed by converting the polymerized precursor to a copolymer containing M¹ and M² as defined above. Any of the types of CP copolymers described above may be incorporated into the star polymer using the above-described process.

Step (ii) of the synthetic method includes functionalizing SP_{c} [symbol] L¹-CP-L′-Rₛ]ₙ with a first reactive functional group R¹ to form the following structure: SP_{c} [symbol] L¹-CP-L′-R¹]ₙ, wherein steps (i) and (ii) may be performed simultaneously or sequentially. Typically, the reactive group on Rₛ is displaced by a reactive-containing molecule of the formula R^{f}-L′-R¹, wherein R^{f} is a group reactive with the reactive group on Rₛ to result in substitution of the reactive group in R^{s} with L′-R¹, wherein L′ may be a bond or any of the groups described earlier above for L′. The reactive functional group R¹ is any group that can form a linkage reaction product with another reactive group present on another molecule for the purpose of attaching the other molecule to the star polymer where R¹ is located. The reactive functional group R¹ may be, for example, amine-reactive, carboxy-reactive, thiol-reactive, or a click chemistry reactive group (e.g., an azido or alkynyl group). More specifically, as further discussed below, R¹ should be sufficiently reactive to react with reactive functional groups R² and R³ to form a linkage reaction product A¹.

Step (iii) involves reacting SP_{c} [symbol] L¹-CP-L′-R¹]ₙ with R²-L³-R³ the hydrogel composition containing 1^{st} and 2^{nd} units. R² and R³ are second and third reactive functional groups, respectively, wherein R¹ and R² react to form a reaction linkage product A¹, and R¹ and R³ react to form another reaction linkage product A¹. For example, R¹ may be azidyl and R² and/or R³ may be alkynyl, or R¹ may be alkynyl and R² and/or R³ may be azidyl, or R¹ may be amino or hydroxyl and R² and/or R³ may be isocyanate, alkyl halide, succinimidyl, or carboxyl ester, or R¹ may be isocyanate, alkyl halide, succinimidyl, or carboxyl ester and R² and/or R³ may be amino or hydroxyl, or R¹ may be maleimide or vinylsulfone and R² and/or R³ may be thiol. L³ is a biodegradable or non-biodegradable linker, as described in detail earlier above.

The reaction of SP_{c} [symbol] L¹-CP-L′-R¹]ₙ with R²-L³-R³ results in the hydrogel composition, as described above containing 1^{st} units and 2^{nd} units interconnected with each other. When no intermediate crosslinking step(s) is/are included in step (ii), the hydrogel composition can be represented by the following structure:

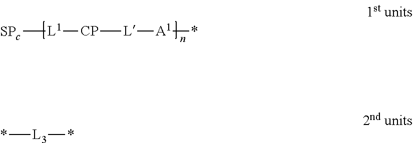

wherein the asterisks (*) in 1^{st} units represent connection points with asterisks in 2^{nd} units, and the hydrogel composition has a multiplicity of 1^{st} and 2^{nd} units.

In some embodiments, step (ii) further includes an intermediate crosslinking step as follows: reacting SP_{c} [symbol] L¹-CP-L′-R¹]ₙ with R^{1′}-L²-R^{2′} to form the following structure: SP_{c} [symbol] L¹-CP-L′-A¹-L²-R^{2′}]ₙ,

wherein: R¹ is a reactive functional group, as described above; R^{1′} is a reactive functional group reactive with R¹ to form a reaction product A¹; and L² is a linker containing at least one carbon atom and optionally one or more heteroatoms selected from O, N, and S, which may be selected from any of the types of linkers described above for L¹ and L′; and R^{2′} is a reactive functional group reactive with R² to form a reaction product A², and R^{2′} is a reactive functional group reactive with R³ to form a reaction product A². A² may independently be selected from any of the reaction products given above for A¹.

Thus, following the intermediate crosslinking step in step (ii), SP_{c} [symbol] L¹-CP-L′-A¹-L²-R^{2′}]ₙ is reacted with R²-L³-R³ to form the hydrogel composition containing 1^{st} and 2^{nd} units having the following structures:

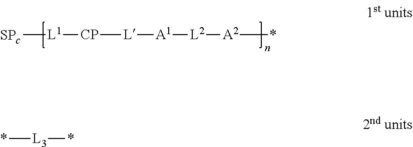

wherein the asterisks (*) in 1^{st} units represent connection points with asterisks in 2^{nd} units, and the hydrogel composition has a multiplicity of 1^{st} and 2^{nd} units.

In the same manner, a second intermediate crosslinking step may be included to result in a second, independently selected L²-A² linkage connected to the first L²-A-linkage. Similarly, a third intermediate crosslinking step may be included to result in a third, independently selected L²-A² linkage connected to the second L²-A² linkage. When more than one intermediate crosslinking step is included, each instance of L² and A² is independently selected. Thus, L² and A² may be the same or different when more than one intermediate L²-A² linkage is present.

To include the possibility of two or three intermediate crosslinking steps in the above described synthetic method, the 1^{st} units of the hydrogel composition can be conveniently expressed as:

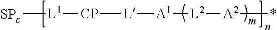

as provided earlier above, wherein m is 0, 1, 2, or 3. When m is 0, no intermediate crosslinking step is included. When m is 1, a single intermediate crosslinking step (and thus, a single L²-A² linkage) is included. When m is 2, two intermediate crosslinking steps (and thus, two independently selected L²-A² linkages) are included. When m is 3, three intermediate crosslinking steps (and thus, three independently selected L²-A² linkages) are included.

In another aspect, the present disclosure is directed to a method of culturing and containing cells. In the method, cells are combined with any of the hydrogel compositions described above under conditions to promote cell survival, maintenance, cell product production, or cell growth. The cell can be any type of cell, including, for example, stem cells, stem cell-derived cells and organoids, spheroids, cancer organoids, healthy organoids, diseased organoids, pancreatic islets, neutrophils, monocytes, T cells, and NK cells. The cell is not limited to any of the specific cell types disclosed herein unless otherwise specified. In some embodiments, the cells are maintained in the hydrogel composition after culturing and/or growth. In some embodiments, the cells are released from the hydrogel composition after culturing, containment, or growth. In some embodiments, the method results in production of biological tissue, such as spheroids and organoids, and/or biological products derived from the cells, such as extracellular vesicles. In some embodiments, continuous exposure of a Rho/ROCK inhibitor is not required (i.e., is not included) for cell survival or growth.

In some embodiments, the released biological tissue is implanted into a living organism under conditions that are non-degradable or MMP-degradable. In other embodiments, any of the hydrogel compositions described above is implanted into a living organism. The hydrogel composition being implanted may or may not contain cells when implanted. The hydrogel composition being implanted may function as, for example, protection of beta cells for sustained insulin release (hydrogel containing cells) or for extended drug release from a hydrogel depot; the creation of an empty scaffold in which native cells can invade and remodel; or for use as a functional tissue substitute such as for cartilage tissue replacement (cell free hydrogel).

Examples have been set forth below for the purpose of illustration and to describe the best mode of the invention at the present time. However, the scope of this invention is not to be in any way limited by the examples set forth herein.

### Examples

### Overview

Human pluripotent stem cells (hPSCs), including induced pluripotent stem cells (iPSCs) and embryonic stem cells (ESCs), exhibit remarkable potential for diverse biomedical applications. However, conventional matrixes used for 2D hPSC expansion often have severe limitations due to their animal origin and undefined complexity. While 3D zwitterionic scaffolds have been studied for supporting the growth of suspension hematopoietic stem and progenitor cells, the use of zwitterionic hydrogels for the 3D culture of adherent stem cells like iPSCs has been significantly impeded by several challenges, including limited hydrolytic stability, poor functionality, slow gelation, and inefficient synthesis methods.

Herein is presented a highly efficient method for synthesizing functional poly(carboxybetaine) (PCB), which offers access to star-shaped zwitterionic polymers with superior hydrolytic stability, excellent functionality, and tunable stiffness under physiological conditions. The developed PCB hydrogel system, incorporating an enzyme-degradable crosslinker and cell-adhesive peptide, permits direct encapsulation and release of hPSCs in a user-friendly manner. The 3D culture of hPSCs using the present PCB hydrogel provides robust cell expansion which results in high yield, excellent pluripotency, and significant differentiation capabilities, all of which highlights the potential of this newly developed zwitterionic hydrogel system as a superior alternative for the expansion and differentiation of various stem cells.

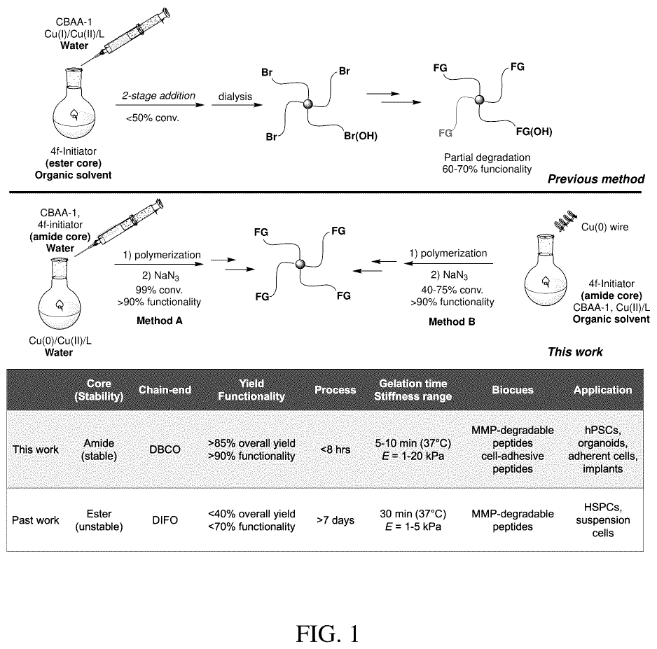

Significant improvements are needed for the design and synthesis of key zwitterionic components in order to provide a broader range of applications. FIG. 1 summarizes the challenges associated with the synthesis of key PCB gel components and hydrogel formation in previous studies and the solutions developed in this work. FIG. 1 also shows some of the distinctions between the conventional art and presently described invention. Some of the challenges associated with PCB gel components of previous studies include: 1) the hydrogel is prone to hydrolytic degradation due to the presence of the ester linkage embedded in the polymer core, thus leading to inconsistent results in long-term cell culture; 2) the degree of chain-end functionalization is low (<70%), thus making it difficult to incorporate sufficient bioactive molecules that are necessary for the culture of adherent cells such as hPSCs; 3) the hydrogels prepared are generally soft (<5 kPa) due to structural defects as a result of the low degree of chain-end functionalization, thus hindering them in applications where a stiffer matrix is preferred; 4) the rate of gelation is typically relatively slow (>30 min), thus resulting in cell settling and incomplete network formation; 5) conventional methods used for synthesizing the functional PCB are generally tedious (>7 days), inefficient, and low-yield (<40% overall yield).

To address these issues, herein is reported a highly efficient method for synthesizing well-defined functional PCBs with superior hydrolytic stability, excellent degree of chain-end functionalization, and the capability of forming hydrogels of tunable stiffness smoothly under physiological conditions. Paired with an enzyme-degradable crosslinker, the presently described PCB hydrogel system is user friendly for direct hPSC encapsulation and release and can be used for robust 3D cell expansion of iPSCs using the PCB hydrogel with an exceptional rate of expansion, high yield, excellent pluripotency, and retained differentiation capabilities.

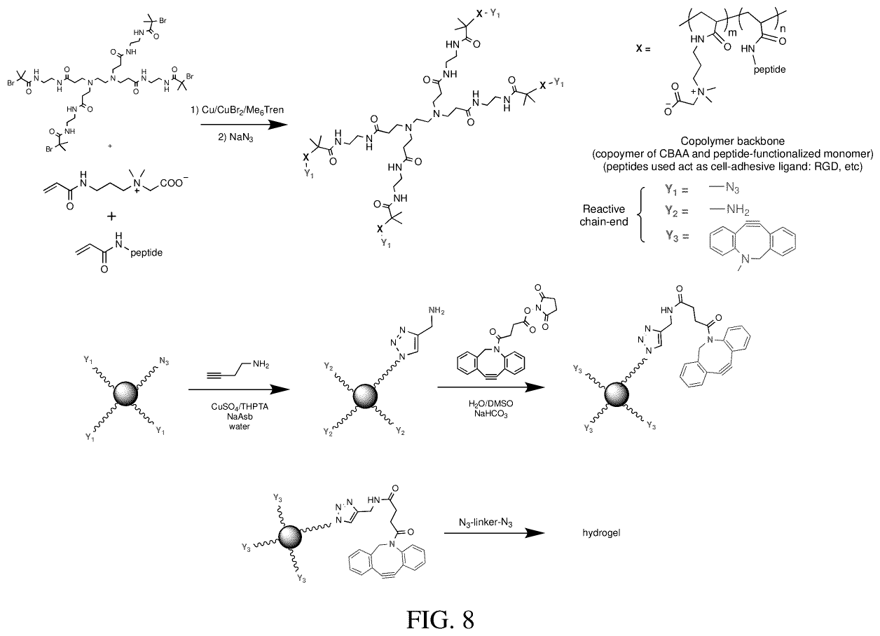

In some embodiments, the present disclosure is directed to the preparation of a representative functionalized zwitterionic copolymer that contains end groups (i.e., azido groups) that permit efficient post-polymerization transformation to other reactive groups to provide crosslinkable polymers useful for forming hydrogels of the invention. Carboxybetaine acrylamide monomer (CBAA) is copolymerized with N-acryl peptides that serve to promote cell-adhesion. The resultant polymer is a random polymer of CBAA and N-acryl peptides. The overall process is shown in FIG. 8.

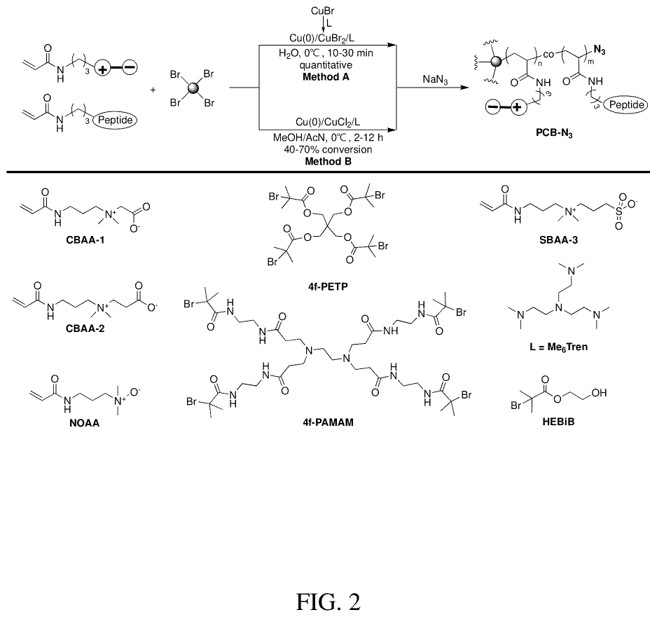

In other embodiments, the present disclosure is directed to an improved synthesis of various linear and 4-arm poly(zwitterionic acrylamide)s, as schematically shown in FIG. 2. The structures of monomers (CBAA-1, CBAA-2, SBMA-3, NOAA (aka.TMAO), initiators (4f-PETP, 4f-PAMAM, HEBiB) and ligand (Me₆Tren) used in this work are shown in FIG. 2. Methods A and B are shown. Method A is well suited when a water-soluble initiator such as 4f-PAMAM and HEBiB is used while method B is more suitable when a hydrophobic initiator is used.

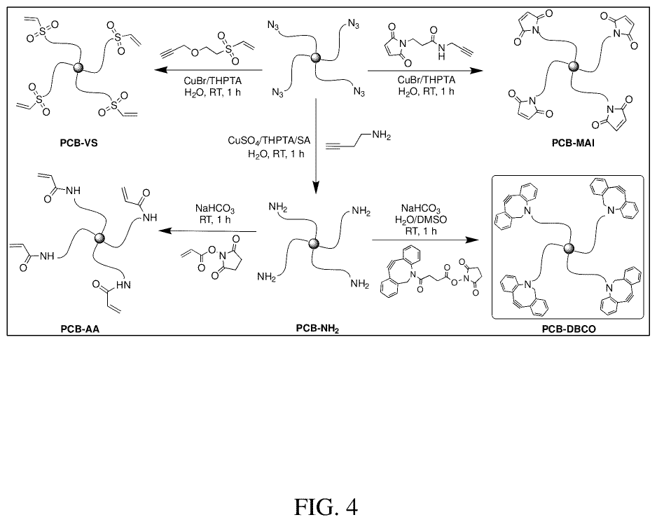

In other embodiments, the present disclosure is directed to a method of post-polymerization modification of 4-arm azide-terminated PCB (PCB-N₃), as schematically shown in FIG. 4. Using this method, star-shaped end-functionalized PCB can be synthesized quantitatively by reacting an alkyne-containing bifunctional molecule with PCB-N₃ or through an amide coupling reaction of PCB-NH₂ with a bifunctional NHS ester.

Synthesis of 4f-PAMAM Initiator

PAMAM-G₀-(NH₂)₄ (20 wt % solution in methanol, 1 mL, purity <86%) was transferred to a round-bottom flask and evaporated under vacuum at 35 °C to give dry starting material (179 mg dry semisolid, 0.35 mmol; 1.4 mmol NH₂). The flask was then placed under the protection of N₂ and 10 mL dry DMF was added and the mixture was slightly warmed up to allowed PAMAM to be dissolved. After dissolution, the flask was placed in an ice bath, and triethylamine (0.30 mL, 2.2 mmol, 1.6 equiv.) was added to the mixture followed by slow addition of α-bromoisobutyrate (0.26 mL, 2.1 mmol, 1.5 equiv.). Upon full addition, the mixture was stirred at 0 °C for another 1 h and allowed to warm up to room temperature and stirred for 16 hours. After the reaction, the mixture was concentrated to dryness and purified by column chromatography twice (Biotage® Sfar Bio C18 D cartridge, 25 g) using water (0.1% TFA, solvent A) and acetonitrile (0.1% TFA, solvent B) as the eluents (10% B to 40% B, 15 min). After lyophilization, the final product was obtained as a white solid (TFA salt, 228 mg, 49% isolated yield). ¹H NMR (500 MHz, D₂O, δ): 3.82 (s, 4H, core CH₂), 3.54 (t, J=6.2 Hz, 2H, NCH₂CH₂CO), 3.43-3.31 (m, 16H, NHCH₂CH₂NH), 2.83 (t, J=6.3 Hz, 8H, CH₂CO), 1.91 (s, 24H, CH₃). ¹³C NMR (126 MHz, D₂O, δ): 174.86, 171.86, 59.73, 50.73, 47.78, 38.98, 38.62, 30.59, 28.44. MS (ESI) m/z: [M+2H]⁺ calcd for C₃₈H₆₈Br₄N₁₀O₈, 557.1; found, 557.2.

### Synthesis of Propargyl 3-Maleimido-Propionamide (MAI-Alkyne)

3-maleimido-propionic NHS ester (538 mg, 2.0 mmol) was suspended in 10 mL of dry dichloromethane in a 20 mL vial, propargyl amine (129 μL, 2.0 mmol) was added to the suspension, and the mixture turned clear within 2 minutes. Triethylamine (282 μL, 2.0 mmol) was then added to the mixture and the reaction mixture was sealed and stirred at room temperature. After 30 min, the mixture was evaporated under vacuum to give the crude product as a viscous orange oil. The crude product was purified by column chromatography (hexanes/ethyl acetate=3:7, v/v) and dried to give the final product as a white solid (417 mg, 91% isolated yield). ¹H NMR (400 MHz, CDCl₃, 6): 6.71 (s, 2H; CH═CH), 5.76 (s, br, 1H; NH), 4.03 (dd, J=5.2, 2.6 Hz, 2H; CH₂NH), 3.85 (t, J=7.2 Hz, 2H; CH₂N), 2.56 (t, J=7.2 Hz, 2H; CH₂CO), 2.23 (t, J=2.6 Hz, 1H; CH═C). ¹H NMR data match the reported values.

### Synthesis of 4-Arm Azide-Terminated 4-Arm PCB (PETP-PCB-N₃) (Method A: Non-Aqueous Polymerization)

CBAA-1 (2.0 g, 9.3 mmol), pentaerythritol tetrakis(2-bromoisobutyrate) (54.4 mg, 0.074 mmol; 0.30 mmol Br), CuCl₂ (40.0 mg, 0.30 mmol) were dissolved in 8 mL of methanol/acetonitrile mixture (1:1, v/v) in a 15-mL falcon tube. Me₆Tren (120 μL, 0.45 mmol) was added, the color of the mixture changed from light sky blue to cyan blue ([M]:[Br]:[CuCl₂]:[L]=31:1:1:1.5). The mixture was then transferred to a Schlenk tube and degassed by freeze-pump-thaw for 3 times. The flask was place in an ice bath and a piece of pre-activated copper wire (20 gauge, 10-20 cm, activated with conc. HCl for 5 min) was dropped into the flask. The mixture was stirred at 0 °C for 4 to 8 hours. NaN₃ (120 mg, 1.85 mmol, N₃/Br=6.2:1) was added and the mixture was stirred for 1 hour at room temperature. The reaction mixture was then diluted with water to about 120 mL and purified by diafiltration using centrifugal filter units having molecular weight cut-off (MWCO) of 10 kDa (at 4000 g, 3 times) to give a concentrated polymer solution. After lyophilization, the product was obtained as a white solid (0.8-1.2 g, 40%~60% isolated yield, Mₙ (NMR)=13~18 kDa).

### Synthesis of 4-Arm Azide-Terminated PCB (PAMAM-PCB-N₃) (Method B: Aqueous Polymerization)

CBAA-1 (2.0 g, 9.3 mmol) and 4f-PAMAM (62.7 mg, 0.0467 mmol; 0.187 mmol Br) were dissolved in 8 mL of water/methanol mixture (7:1, v/v) in a falcon tube. The mixture was degassed by N₂ purging for over 15 min at 0 °C. In a 25 mL Schlenk flask, CuBr (26.7 mg, 0.187 mmol) was added to 2 mL of water and the mixture was degassed by freeze-pump-thaw (three times). Me₆Tren (50 μL, 0.187 mmol) was added, the mixture turned blue followed by immediate formation of dark precipitate (metallic copper). After the catalyst solution was stirred for 15 min at 0 °C, the solution containing the monomer and the initiator was transferred to the Schenk flask via a nitrogen flushed syringe ([M]:[Br]: [CuBr]: [L]=50:1:1:1) and the reaction mixture was allowed to stir at 0 °C for 10 minutes during which a significant increase in viscosity could be observed. After the polymerization, NaN₃ (18 mg, 0.28 mmol, N₃/Br=1.5) was added and the mixture was stirred for another 1 hour at room temperature. The mixture was diluted with water to about 120 mL, centrifuged to remove metallic copper, and purified by diafiltration using centrifugal filter units (MWCO 10 kDa, 4000 g, 3 times). The concentrated product solution was freeze-dried to give a white solid as a final product (1.90 g, 95% yield, Mₙ (NMR)=55~65 kDa).

Synthesis of NH₂-Terminated 4-Arm PCB (PCB-NH₂)

To 10 mL of an aqueous solution of PCB-N₃ (10 wt %), CuSO₄/THPTA stock solution (200 μL, Cu/THPTA=1:1.1, 100 mM), 3-butyn-1-amine (alkyne/N₃=1.5:1), and acetic acid (HOAc/alkyne=1:1) were added successively. The mixture was then sealed with a screw cap (fitted with a septum) and 100 μL of freshly made sodium ascorbate solution (200 mM, SA/Cu=2:1) was added via syringe during which the color of the mixture turned from blue to colorless. The mixture was allowed to stir at room temperature for 1 hour before being diluted with water and purified by centrifugal dialysis using a centrifugal filter unit (MWCO 10 kDa, 4000 g, 3 times). The concentrated product solution was lyophilized to give a white solid as a final product (0.95 g, 95% yield, end-group fidelity 90-95%).

Synthesis of RGD-Containing N₃-Terminated 4-Arm PCB (PCB-RGD-N₃)

Cyclic peptide, c(RGDyK) (TFA salt, 25 mg, 0.029 mmol) was dissolved in 1 mL of NaHCO₃ buffer (0.1 M), and N-acryloxysuccinimide (5.3 mg, 0.031 mol) was added. The mixture was stirred at room temperature for 1 h to reach 98+% conversion, as determined by LC-MS. CBAA-1 (1.2 g, 5.6 mmol) and 4f-PAMAM (36.9 mg, 0.028 mmol) were dissolved in 5 mL of water/ethanol mixture (9:1, v/v) in a falcon tube and the peptide solution was added. The mixture was degassed by N₂ purging for over 15 min at 0 °C. In a 25 mL Schlenk flask, CuBr (15 mg, 0.10 mmol) was added to 2 mL of water and the mixture was degassed by freeze-pump-thaw three times. Me₆Tren (28 μL, 0.10 mmol) was added, the mixture turned blue, and dark precipitate (metallic copper) formed immediately. After the catalyst solution was stirred for 15 min at 0 °C, the solution containing the monomer, the initiator, and the peptide was transferred to the Schenk flask via a nitrogen flushed syringe ([M]:[Br]:[CuBr]:[L]=50:1:1:1), and the reaction mixture was allowed to stir at 0 °C for 10 min. After the polymerization, NaN₃ (65 mg, 1.0 mmol) was added and the mixture was stirred for another 1 hour at room temperature. The mixture was diluted with water to about 60 mL, centrifuged to remove metallic copper, and purified by diafiltration using centrifugal filter units (MWCO 10 kDa, 4000 g, 3 times). The concentrated product solution was freeze-dried to give a white solid as a final product (1.1 g, 92% yield, Mₙ (NMR)=66 kDa, RGD=0.40 mol %).

### Synthesis of MAI- and VS-Terminated 4-Arm PCB (PCB-MAI and PCB-VS)

To 10 mL of an aqueous solution of PCB-N₃ (10 wt %), THPTA (8.7 mg, 0.020 mmol) and MAI-alkyne or 3-(2-(vinylsulfonyl)ethoxy)prop-1-yne (VS-alkyne) (alkyne/N₃=2:1) were added successively. The mixture was then sealed with a screw cap (fitted with a septum) and purged with N₂ for 15. After purging, CuBr (2.8 mg, 0.020 mmol) was quickly added to the vial, and the mixture was sonicated for 2 minutes until the mixture was homogeneous. The mixture was then stirred at room temperature for 1 hour before being diluted with water and purified by centrifugal dialysis using a centrifugal filter unit (MWCO 10 kDa, 4000 g, 3 times). The concentrated product solution was lyophilized to give a white solid as a final product (0.95 g, 95% yield, end-group fidelity 90-95%).

### Synthesis of DBCO-Terminated 4-Arm PCB (PCB-DBCO)

PCB-NH₂ (1.8 g, DP=284, end-group fidelity=96%, 0.11 mmol NH₂) was dissolved in 9 mL of NaHCO₃ buffer (0.1 M) followed by addition of 8 mL of DMSO. DBCO-NHS solution (60 mg in 1 mL DMSO, 0.15 mmol) was added dropwise while the mixture was stirred vigorously. Upon complete addition of DBCO-NHS, the reaction mixture became cloudy and it was stirred at room temperature for 1 hour. After the reaction was complete, the mixture was filtered through 0.45 μm nylon filter, diluted with water, and purified by diafiltration using centrifugal filter units (MWCO 10 kDa, 4000 g, 4 times). The concentrated polymer was lyophilized to give the final product as a white solid (1.6 g, 89% isolated yield, DP=282, DS=95%).

### Synthesis of DBCO-Terminated 4-Arm PEG (PEG-DBCO)

NH₂-terminated (HCl salt) 4-arm PEG (41 kDa, 1.0 g, 0.098 mmol NH₂) was dissolved in 5 mL of dry dichloromethane. Triethylamine (28 μL, 0.20 mmol) and DBCO-NHS (48 mg, 0.12 mmol, 1.2 equiv.) were successively added to the solution. The reaction mixture was stirred at room temperature for 1 hour. After the reaction, the mixture was precipitated into 100 mL cold ether, filtered, and rinsed to give the crude product as a white solid (0.98 g). The crude product was purified by centrifugal dialysis using filter units (MWCO 10 kDa, 4000 g, 3 times) and lyophilized to give the final product as a white solid (0.81 g, 79% yield, DS=97%).

### Peptide Synthesis

Modified MMP-degradable peptide, EK_{N3}GPQGIWGQK_{N3}E was synthesized following the standard SPPS protocol using a microwave peptide synthesizer. The synthesis was carried out on a 0.25 mmol scale using Fmoc-rink amide MBHA (100-200 mesh) as the resin, DIC as the coupling reagent, and OxymaPure as the coupling additive. Following the synthesis, the resin was rinsed, dried, and treated with 5 mL of a peptide cleavage cocktail (90% TFA, 2.5% water, 2.5% thioanisole, 2.5% phenol, 2.5% 1,2-ethanedithiol) for 2 hours. The peptide was then precipitated into cold ether, washed twice with ether, and dried to give a crude product. The crude peptide was purified by HPLC (Agilent Prep-C18 30×100 mm, 5% B to 35% B, 15 min). After lyophilization, the final product was obtained as a white solid (114 mg, 30% yield) with 98+% purity. TFA was exchanged by freeze-drying the peptide solution (20 mL, 10 mM HCl) twice.

### Hydrogel Preparation and Stiffness Measurement

For each composition, a pre-chilled 4-arm DBCO-terminated PCB (PCB-DBCO) solution (8-16% w/v in water) was mixed with a stochiometric amount of 1,8-diazido-3,6-dioxaoctane (N₃—PEG₃-N₃) (5 mg/mL in water), and the mixture was quickly vortexed and centrifuged at 10000 g for 1 minute at 0 °C. The solution was then carefully transferred into a glass mold made from two glass slides (pretreated with Rain-X) and one 800 μm-thick PTFE spacer. The glass mold was sealed by parafilm and placed in an oven thermostated at 40 °C for 30 min so that the gelation was complete. The glass slides were then removed from the oven and carefully separated to release the hydrogel into a culture dish full of water. After equilibration at room temperature for 48 hours, disk-shape specimen were created using an 8 mm biopsy punch, and the Young's modulus was measured using a universal testing system (10 N load cell, 3%-8% strain).

### Gelation Kinetics

For each composition, a pre-chilled PCB polymer solution was quickly added to crosslinker (N₃—PEG₃-N₃, 5 mg/mL in water), vortexed, and loaded onto a DHR-3 rheometer with parallel plate geometry (20 mm Aluminum Peltier plate, 500 μm gap) conditioned at 37 °C. After the gap was closed, a solvent trap was added and the shear storage modulus of the PCB hydrogels was measured at an oscillation frequency of 1 Hz and a strain of 1% at 37 °C.

### Cell Culture

The iPSCs were cultured on 6 well plates coated with Matrigel® (Corning®, 354230) in mTeSR™ Plus stabilized cell culture medium supplemented with 50 μg/mL Normocin™ antimicrobial reagent. Cells were passaged when confluent with Accutase® cell detachment solution for 6-8 min at 37 °C and replated on 6 well plates with Matrigel® and mTeSR™ Plus medium with 50 μg/mL Normocin™ supplemented with 10 μM Rho/ROCK inhibitor for the first 24 hours. Medium was changed daily.

Encapsulation and Release of Cells within Hydrogels

iPSCs on Matrigel® were incubated with Accutase® at 37 °C for 6-8 min and dissociated into single cells. Dissociated cells were centrifuged at 300 g for 5 min and a cell density of 10,000 cells/μL hydrogel was used for encapsulation. A polymer molecular weight of 60 kDa and final polymer concentration of 8 wt % was used as this results in a hydrogel with a Young's modulus of ~1.5 kPa which matches the modulus during embryogenesis. The cell pellet was first resuspended in PCB-RGD-DBCO polymer. Then, a bis(azide)-functionalized MMP-degradable peptide (EK_{N3}GPQGIWGQK_{N3}E) was added and the cell solution was mixed thoroughly. This solution was plated as 20 μL droplets to each well of a 24 well plate or a 6 well plate. After 10 minutes in a 37 °C incubator, homogenous cell-laden PCB hydrogels were formed. Hydrogels were washed once with mTeSR™ Plus media supplemented with 10 μM Y-27632 ROCK inhibitor and then this media was added to cover the gels. mTeSR™ Plus medium with 10 μM Y-27632 was used for daily media changes for the duration of the experiment as this has been shown to enhance cell proliferation in 3D environments (Y. Lei et al., Proceedings of the National Academy of Sciences 2013, 110, E5039). To release iPSCs from the PCB hydrogel at desired timepoints, the hydrogel was incubated with 0.1% collagenase for 35 minutes at 37 °C. Cells were then collected for various assays.

### Viability Assay

At designated timepoints, iPSCs were released from PCB hydrogels using collagenase by known methods. Spheroids were collected with wide bore pipette tips and allowed to settle by gravity for 15 min. The supernatant was carefully removed, and Calcein AM (to stain live cells) and BOBO-3™ Iodide (to stain dead cells) were added to the cell pellet in a 1:1 dilution in mTeSR™ Plus media for final dye concentrations of 2 μM and 0.25 μM, respectively. Hoechst® 33342 was also added 1:1000 to the dye mixture to stain the nucleus of all cells for easier quantification. After incubating the cells for 30 min. on an orbital shaker at 50 rpm, the spheroids were allowed to settle by gravity for 15 min. Subsequently, the supernatant was carefully removed, and the spheroids were resuspended in 1×PBS using wide bore pipette tips. This was then transferred to a glass bottom 96 well plate for bright field and fluorescent imaging on a Spinning Disk Confocal Microscope. Three images were taken for each biological replicate in different locations of each well as technical replicates. Images were processed and viability was quantified.

### Cell Proliferation

At desired timepoints, iPSCs were released from PCB hydrogels using collagenase as previously described. Cells were collected and centrifuged at 300 g for 5 minutes. Cells from day 0 and 1 proceeded to counting. Spheroids day 3+ were first resuspended in Accutase™ at 37 °C for 15 min until spheroids were dissociated into single cells. Dissociated cells were centrifuged at 300 g for 5 min and the supernatant was carefully removed. The cell pellets from all timepoints were resuspended in mTeSR™ Plus media and counted on a Cellometer® Auto 2000 cell viability counter. Total cell count was recorded to measure cell growth. Fold change in expansion from day 0 was calculated by dividing the total cell count measured on days 1, 3, 5, and 7 by the total cell count measured on day 0.

### Germ Layer Differentiation

For germ layer differentiation outside the PCB hydrogel, iPSC spheroids were released from PCB hydrogels after 5 days of culture as previously described and dissociated to single cells using Accutase™. Cells were then plated onto phenol red-free Matrigel® coated 24 well plates for RNA isolation and glass bottom 24 well plates for immunofluorescence staining. Cells were then differentiated to the ectoderm, mesoderm, and endoderm germ layers by the STEMdiff™ Trilineage Differentiation Kit, a functional assay kit for in vitro directed differentiation of human ES and iPS cells to assess pluripotency. The medium was changed daily. For germ layer differentiation inside the PCB hydrogel, iPSCs were encapsulated in the PCB hydrogel by known methods and cultured in mTeSR™ Plus medium supplemented with 50 μg/mL Normocin™ and 10 μM Rho/ROCK inhibitor for the first 24 hours. Cells were then differentiated to the ectoderm, mesoderm, and endoderm germ layers by the STEMdiff™ Trilineage Differentiation Kit. Medium was changed daily.

### Karyotyping

iPSCs were cultured in the PCB hydrogel for 5 days, released as previously described using collagenase, and dissociated to single cells using Accutase™. These cells were then plated onto Matrigel® coated T25 flasks. The flasks then underwent karyotype analysis.

Immunofluorescence Staining for iPSC Spheroids

After 5 days of culture inside the PCB hydrogel, iPSC spheroids were released as previously described, collected with wide bore pipette tips into 1.5 mL tubes, and allowed to settle by gravity for 15 min. Spheroids were washed once with 1×PBS and then fixed with 4% formaldehyde for 1 hr at 37 °C followed by two washes with 1×PBS. For OCT4 and NANOG staining, the spheroids were permeabilized with 0.5% Triton X-100 (in 1×PBS for 30 min. at room temperature (RT) on a plate shaker at 50 rpm. Spheroids were blocked with 0.1% Triton X-100 and 10% goat serum in 1×PBS for 1 hr at RT on a plate shaker at 50 rpm. For SSEA-3 and SSEA-4 staining, the spheroids were left unpermeabilized and blocked with only 10% goat serum in 1×PBS for 1 hr. The spheroids were then incubated with primary antibodies against OCT4 at 1:200, NANOG at 1:2000, SSEA-3 at 1:200, and SSEA-4 at 1:250 in respective blocking buffers overnight at RT on a plate shaker at 50 rpm. After three washes with 0.1% Tween-20 in 1×PBS for OCT4 and NANOG samples and three washes with 1×PBS for SSEA-3 and SSEA-4 samples, spheroids were incubated with secondary antibodies Goat Anti-Rabbit IgG Alexa Fluor®647 at 1:1000, Goat Anti-Mouse IgG Alexa Fluor®555 at 1:1000, Goat Anti-Rat IgM Alexa Fluor®488 at 1:1000, and Hoechst® 33342 at 1:1000 in respective blocking buffers for 3 hours at RT on a plate shaker at 50 rpm. After three washes with respective washing buffers, spheroids were added to glass slides, mounted using mounting media, and allowed to dry overnight. Slides were imaged using a 40× objective with 1.6× zoom using a confocal microscope, and images were analyzed using an image processing package.

### Immunofluorescence Staining for Germ Layer Differentiation Outside PCB Hydrogel

After 5 days of differentiation for endoderm and mesoderm differentiated samples and 7 days of differentiation for ectoderm differentiated samples, cells were washed with 1×PBS, fixed with 4% PFA for 20 min at RT, and washed twice with 1×PBS. Samples were permeabilized with 0.5% Triton X-100 in 1×PBS for 15 min. at RT on a plate shaker at 50 rpm. Samples were blocked with 0.1% Triton X-100 and 10% goat serum in 1×PBS for 30 min. at RT on a plate shaker at 50 rpm. The samples were then incubated with primary antibodies against PAX6 at 1:400, Nestin at 1:1000, FOXA2 at 1:800, SOX17 at 1:100, Brachyury at 1:400, and α-SMA at 1:100 in blocking buffer overnight at 4 °C. After three washes with 0.1% Tween-20 in 1×PBS, samples were incubated with secondary antibodies Goat Anti-Rabbit IgG Alexa Fluor®647 at 1:1000, Goat Anti-Mouse IgG Alexa Fluor®555 at 1:1000, and Hoechst® 33342 at 1:1000 in blocking buffer for 2 hours at RT on a plate shaker at 50 rpm. Samples were washed three times with 0.1% Tween-20 in 1×PBS and stored in 1×PBS with 0.02% sodium azide. Samples were imaged with a 40× objective using a confocal microscope, and images were analyzed.

### Immunofluorescence Staining for Germ Layer Differentiation Inside PCB Hydrogel

After 5 days of differentiation for endoderm and mesoderm differentiated samples and 7 days of differentiation for ectoderm differentiated samples, germ layer organoids were released from the PCB hydrogel as previously described using collagenase, collected with wide bore pipette tips into 1.5 mL tubes, and allowed to settle by gravity for 15 min. Samples were washed once with 1×PBS and then fixed with 4% formaldehyde for 1 hr at 37 °C followed by two washes with 1×PBS. Samples were permeabilized with 0.5% Triton X-100 in 1×PBS for 30 min. at RT on a plate shaker at 50 rpm. Spheroids were blocked with 0.1% Triton X-100 and 10% goat serum in 1×PBS for 1 hr at RT on a plate shaker at 50 rpm. The samples were then incubated with primary antibodies against PAX6 1:400, Nestin at 1:1000, FOXA2 at 1:800, SOX17 at 1:100, Brachyury at 1:400, and α-SMA at 1:100 in blocking buffer overnight at RT on a plate shaker at 50 rpm. After three washes with 0.1% Tween-20 in 1×PBS, organoids were incubated with secondary antibodies Goat Anti-Rabbit IgG Alexa Fluor®647 at 1:1000, Goat Anti-Mouse IgG Alexa Fluor® 555 at 1:1000, and Hoechst® 33342 at 1:1000 in blocking buffer for 3 hours at RT on a plate shaker at 50 rpm. After three washes with 0.1% Tween-20 in 1×PBS, organoids were added to glass slides, mounted using mounting media, and dried overnight. Slides were imaged using a 40× objective with 1.6× zoom on a confocal microscope, and images were analyzed.

### RT-qPCR

RNA was isolated for pluripotency samples, endoderm differentiated samples, and mesoderm differentiated samples after 5 days of culture while RNA was isolated for ectoderm differentiated samples after 7 days of culture. RNA was isolated using a commercial kit following the manufacturer's instructions at designated timepoints. Reverse transcription of RNA into complementary DNA (cDNA) was performed using the a commercial kit. Generated cDNA was diluted 1:5 in nuclease-free water for qPCR. Real-time qPCR was obtained using a commercial system by following the manufacturer's instructions. Human GAPDH was used as the reference gene for each sample and fold changes in gene expression (2^{-ΔΔCt}) were analyzed in reference to iPSCs cultured on Matrigel® for pluripotency genes and undifferentiated iPSCs for germ layer genes.

### Alkaline Phosphatase (AP) Assay

After 5 and 7 days of culture, iPSC spheroids were released from PCB hydrogels as previously described and were fixed with 4% PFA for 1 hr at 37 °C. Spheroids were stained for 15 min. using an alkaline phosphatase detection kit on a glass bottom 96 well plate, and the entire wells were imaged. Counts for AP+ and AP− spheroids were quantified.

### Statistical Analysis

Material characterization data were shown as mean value±standard deviation. Biological data were shown as mean value±standard error of mean. Commercial software was used for statistical analysis. Student's t-test was used to assess statistical significance between groups, and p<0.05 was considered statistically significant.

### Polymer Toxicity Assay

iPSCs were encapsulated in PCB/PEG hydrogels with different chain-ends (MAI and DBCO) by combining the polymer with their respective nondegradable crosslinker and the iPSCs on 96 well plates. Samples were assayed for viability after 24 hours of culture in the hydrogels. To evaluate viability, the media above the gel was carefully removed, and Calcein AM to stain live cells and BOBO-3 Iodide to stain dead cells were added on top of the hydrogel in a 1:1 dilution in mTeSR™ Plus media for final dye concentrations of 2 μM and 0.25 M, respectively. Hoechst® 33342 was also added 1:1000 to the dye mixture to stain the nucleus of all cells for easier visualization and quantification. After staining the gels for 1 hour at RT to allow for diffusion of the dyes into the gels, the dye supernatant was carefully removed, and 1×PBS was added. The samples proceeded to fluorescent imaging on a cell imaging multi-mode reader, and images were processed.

### Results and Discussion

Previous work used pentaerythritol tetrakis(2-bromoisobutyrate) (4f-PETP) as the initiator for synthesizing 4-arm poly(carboxybetaine acrylamide) (PCB). As a result, the polymer featured a core susceptible to hydrolysis. Moreover, due to significant differences in solubility between the initiator and monomer, the polymerization was conducted in a delicate manner to avoid phase separation, thus resulting in low yields and variable outcomes (see FIG. 1). A significant objective was to improve the synthesis of PCB polymer and ensure the efficient installation of appropriate end-functionality, which is crucial for generating the PCB hydrogel for hPSC expansion and differentiation. As further discussed below, the present results demonstrate the capability of the newly developed PCB hydrogel to directly encapsulate and release hPSCs in a user-friendly manner. The present results also demonstrate high cell viability, high fold expansion, and high cell yield with hPSCs expanded over 7 days in the PCB hydrogel. The results also demonstrate excellent and homogenous pluripotency in hPSCs expanded in the PCB hydrogel. The results show that these hPSCs retain the ability to undergo multi-lineage differentiations after expansion. The present work also explores the use of the PCB hydrogel as a platform for 3D differentiation. Collectively, the following data and results demonstrate the potential for this hydrogel system to replace conventional hydrogels as a next-generation synthetic scaffold for stem cell expansion, differentiation, and organoid growth.

### Synthesis of Hydrolytically Stable Star-Shaped Zwitterionic Polymers

FIG. 2 shows the improved synthesis of various linear and 4-arm poly(zwitterionic acrylamide)s and the structures of monomers (CBAA-1, CBAA-2, SBAA-3, NOAA), initiators (4f-PETP, 4f-PAMAM, HEBiB) and ligand (Me₆Tren) used in this work. Method A is well suited when a water-soluble initiator such as 4f-PAMAM and HEBiB are used while method B is more suitable when a hydrophobic initiator is used.

Using 4f-PETP as the initiator, the living radical polymerization (LRP) of CBAA-1 proceeded smoothly in methanol/acetonitrile (1/1) using Cu(0)/CuCl₂/Me₆Tren catalytic system. See FIG. 2, method B; Table 1, entry 10 at 0 °C. The pure organic solvent system was chosen so that all reactants and products would remain soluble during the reaction.

TABLE 1
Representative examples of Cu(0)-mediated LRP of zwitterionic acrylamides.

| | | | | | | | | | | End- |
|---|---|---|---|---|---|---|---|---|---|---|
| | | | Feed | Time | | | Mₙ | | Mₙ (Xₙ) | group |
| Entry | Initiator | Monomer | ratio | [min] | Method | Conv.^{a)} | [kDa]^{b)} | Ð^{b)} | [kDa]^{a)} | fidelity^{c)} |
| 1 | 4f-PAMAM | CBAA-1 | 100 | 7 | A^{d)} | 98% | 12.1 | 1.11 | 28.7 | 93% |
| | | | | | | | | | (134) | |
| 2 | 4f-PAMAM | CBAA-1 | 200 | 10 | A | 99% | 20.7 | 1.16 | 51.1 | 94% |
| | | | | | | | | | (238) | |
| 3 | 4f-PAMAM | CBAA-1 | 300 | 10 | A | 99% | 29.2 | 1.24 | 70.6 | 90% |
| | | | | | | | | | (330) | |
| 4 | HEBiB | CBAA-1 | 140 | 30 | A | 99% | 18.0 | 1.10 | 36.4 | 85% |
| | | | | | | | | | (170) | |
| 5 | HEBiB | NOAA | 100 | 10 | A | 99% | 13.9 | 1.18 | 16.9 | 95% |
| | | | | | | | | | (98) | |
| 6 | HEBiB | CBAA-2 | 100 | 10 | A | 99% | 17.4 | 1.29 | 26.7 | ND |
| | | | | | | | | | (117) | |
| 7 | HEBiB | CBAA-2 | 100 | 30 | A | 96% | 15.2 | 1.13 | 23.5 | 94% |
| | | | | | | | | | (103) | |
| 8 | HEBiB | SBAA-3 | 100 | 10 | A | 58% | 10.2 | 1.98 | 23.9 | ND |
| | | | | | | | | | (86) | |
| 9 | HEBiB^{f)} | SBAA-3 | 100 | 10 | A | 99% | 14.0 | 1.08 | 30.6 | 93% |
| | | | | | | | | | (110) | |
| 10 | 4f-PETP | CBAA-1 | 125 | 240 | B^{e)} | 63% | 7.6 | 1.18 | 15.8 | 88% |
| | | | | | | | | | (74) | |
| 11 | 4f-PETP^{g)} | CBAA-1 | 125 | 540 | B | 74% | 10.0 | 1.12 | 28.8 | 75% |
| | | | | | | | | | (135) | |
| 12 | 4f-PAMAM | CBAA-1 | 150 | 540 | B | 41% | 7.2 | 1.21 | 16.4 | 69% |
| | | | | | | | | | (34) | |

^{a)}Conversions and absolute Mₙ (Xₙ) were determined from ¹H NMR;
^{b)}relative Mₙ and Ð were measured by GPC, against narrow PEG standards;
^{c)}end-group fidelity was indirectly determined from ¹H NMR after the polymer was azidated and clicked with propargyl amine or 3-butyn-1-amine, estimated error ± 2.5%; ND = Not Determined;
^{d)}Method A (FIG. 2): aqueous polymerization, 10 or 20% (w/v) monomer, 1 equiv. CuBr relative to Br, 1 equiv. Me₆Tren relative to CuBr, water/methanol (9/1 v/v), 0 °C;
^{e)}Method B (FIG. 2): non-aqueous polymerization, 20% (w/v) monomer, 2 equiv. CuCl₂ relative to Br, 1.5 equiv. Me₆Tren relative to CuCl₂, 5-10 cm Cu(0) wire, 0 °C;
^{f)}1 equiv. NaBr (relative to the monomer) was added;
^{g)}the polymerization was carried out at room temperature.

Despite the slow reaction and incomplete monomer conversion, markedly improved yield (>60%) and end-group fidelity (>85%) were achieved. To tackle the issue of core degradability, a polyamidoamine (PAMAM)-based tetrafunctional initiator, 4f-PAMAM was synthesized and employed for the polymerization using the same method (Table 1, entry 12). However, the polymerization was found to result in significant chain-end breakdown, presumably due to intramolecular nucleophilic attack from the amide groups in the initiator. Fortunately, due to the improved water solubility of 4f-PAMAM, the polymerization could be conducted in an aqueous phase using protocols that were recently established for neutral and anionic acrylamide monomers, in particular the Cu(0)-mediated LRP technique (FIG. 2, method A) (G. R. Jones et al., Angew. Chem. Int. Ed., 57, 10468, 2018; C. Waldron et al., Polymer Chemistry, 5, 57, 2014). Surprisingly, in the presence of the in situ generated Cu(0) and Cu(Me₆Tren)Br₂, the aqueous polymerization of CBAA-1 was found to be very fast, and moreover, near quantitative monomer conversion (>99%) could be reached within 10 minutes with narrow dispersity (1.05<Ð<1.25) and tunable molecular weight (Table 1, entry 1-3). Besides CBAA-1, this technique was found to work well for several other zwitterionic acrylamide monomers that have greater basicity, such as CBAA-2 (pK_{b}~10.7) and NOAA (pK_{b}~9.5), although monomers with a longer spacer between the charges such as CBAA-2 and SBAA-3 require the addition of halide salt to improve the livingness (Table 1, entry 5-9).

By virtue of the high efficiency, it was herein found that water-soluble cell-adhesive peptides, such as c(RGDyK), can be efficiently incorporated (up to 90% efficiency), into the polymer via direct copolymerization with CBAA-1 upon in situ acylation with N-acryloxysuccinimide. The ability to incorporate bioactive molecules via direct polymerization not only permits greater loading capacity but also fully preserves the end-group for gelation, which makes it possible to independently control stiffness and bioactivity of a hydrogel network. While the aqueous polymerization protocol (FIG. 2, method A) has been proven effective for the objectives of this work, the non-aqueous protocol (FIG. 2, method B) may be useful in situations where amphiphilic zwitterionic copolymers are the target.

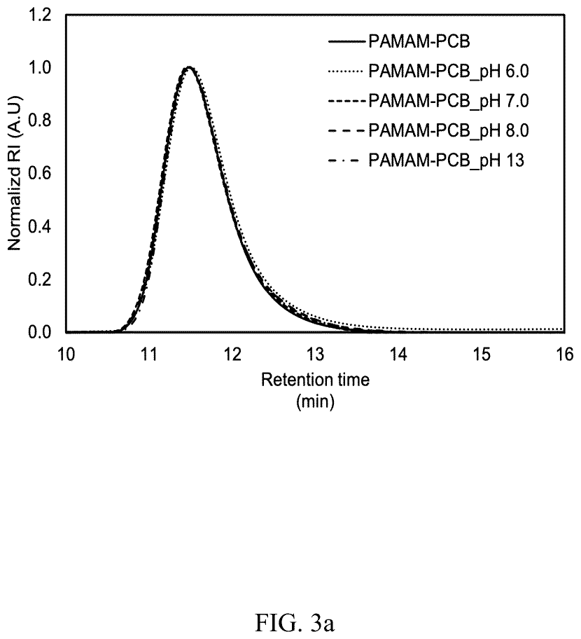

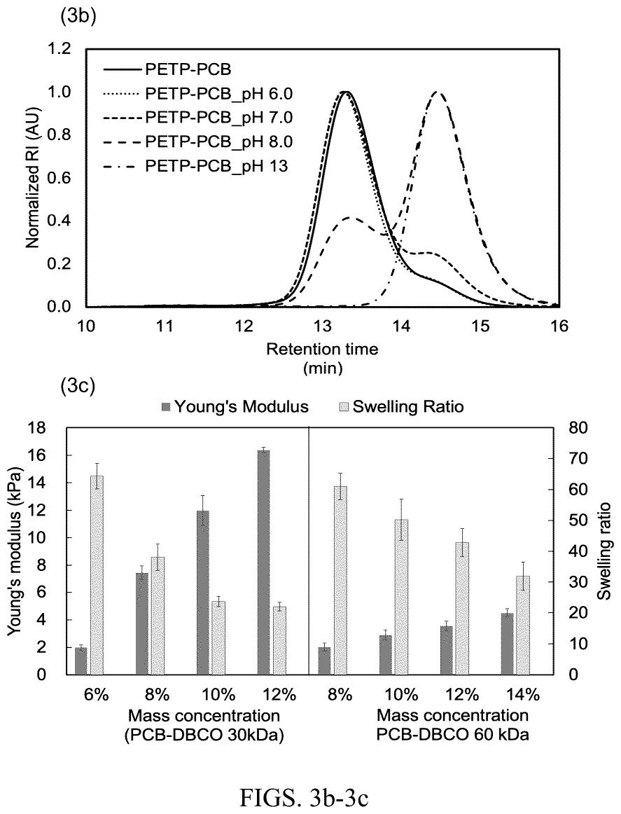

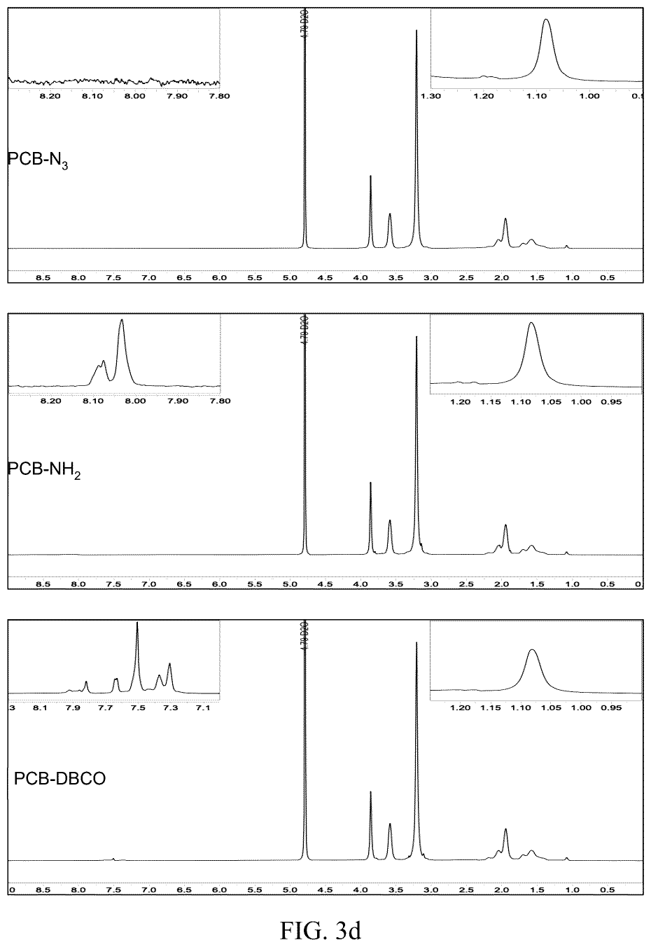

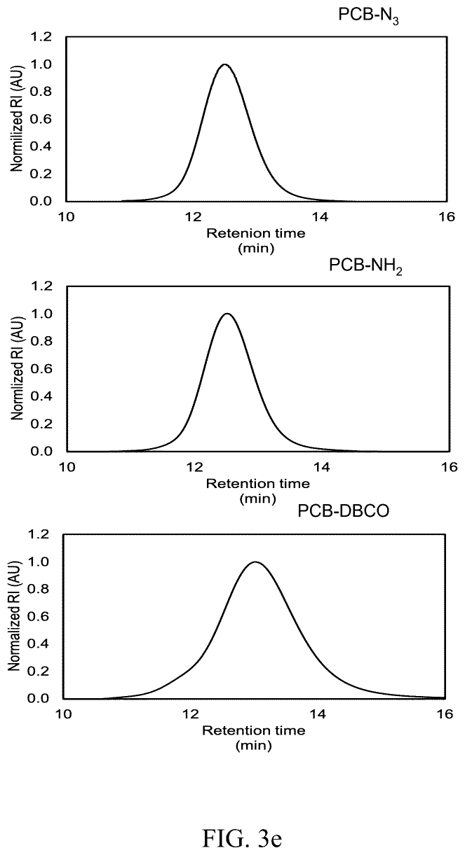

To evaluate the hydrolytic stability of the PAMAM-PCB and PETP-PCB, each polymer was dissolved in PBS buffers with different pHs and incubated at 37 °C. Upon GPC analysis, it was found that PAMAM-PCB is completely stable under all conditions (FIG. 3a). Even under strong alkaline conditions (0.1 M NaOH), no significant change in molecular weight distribution was observed, which confirms its extraordinary hydrolytic stability. In contrast, although PETP-PCB appears to be stable at slightly acidic environment (pH 6.0), it underwent noticeable hydrolysis even under neutral conditions and a slight increase in pH was seen to significantly accelerate the PETP-PCB degradation (FIG. 3b). Under strong alkaline conditions (0.1 M NaOH), PETF-PCB underwent full hydrolysis into small PCB fragments whose molecular weight and dispersity are consistent with the expected value.

Having established the capability of producing hydrolytically stable, star-shaped zwitterionic polymers with excellent yield, the focus shifted to ensuring the efficient installation of appropriate end-functionality, which is critical for the generation of PCB hydrogel with a versatile range of stiffness under conditions that are conducive for cell encapsulation (particularly for hPSCs expansion and differentiation).

### End-Functionalized 4-Arm Poly(Carboxybetaine Acrylamide) (PCB) and its Gelation

The lability of chain-end (Br) is a well-known issue for LRP of acrylamides and the loss of chain-end will eventually give rise to compromised hydrogel properties. As it is often believed that the loss of Br chain-end is due to hydrolysis with or without neighboring group participation, in order to minimize the chain-end loss during and after polymerization, the polymerization time was minimized (Table 1, entry 4 and 11) and had bromine chain-ends directly converted to azido groups via in-situ radical substitution. It was found that, by using a small excess of sodium azide, azide-terminated PCB can be produced rapidly and quantitatively (FIG. 2). To install the final reactive groups that are responsible for gel formation, copper-catalyzed azide-alkyne cycloaddition (CuAAC) was employed to click the azide-terminated PAMAM-PCB and an alkyne-containing bifunctional molecule together to produce the desired end-functionalized PCB featuring maleimide, vinyl sulfone or amine as the end group. See FIG. 4, which is a schematic showing post-polymerization modification of 4-arm azide-terminated PCB (PCB-N₃). The scheme in FIG. 4 shows that star-shaped end-functionalized PCB can be synthesized quantitatively by reacting an alkyne-containing bifunctional molecule with PCB-N3 or through an amide coupling reaction of PCB-NH2 with a bifunctional NHS ester.

It was herein found that the click reaction can be conveniently carried out in water using Cu(II)-THPTA complex as the pre-catalyst and sodium ascorbate (SA) as the reducing agent. Under typical conditions ([N₃]≈20 mM, [Cu/THPTA]=2 mM, 1.5~2 equiv. alkyne to azide, 4 mM SA), the reaction was complete within one hour and the transformation was near quantitative as determined by ¹H NMR (see FIG. 3d). However, prolonged reaction should be avoided as SA is nucleophilic enough to react slowly with electron deficient alkene such as vinyl sulfone and maleimide, and dehydroascorbate (oxidized SA) can gradually react with amine, which results in loss of chain-end functionality. To substantially eliminate these side reactions, CuBr may be used instead, and the reaction is best carried out under an inert atmosphere using degassed solvent. Due to intrinsic reactivity towards organic azide or azide ion, azadibenzocyclooctyne (DBCO) cannot be directly incorporated to PCB-N₃ using click chemistry; instead, DBCO can be efficiently installed via the reaction of PCB-NH₂ intermediate with an amine-reactive DBCO-NHS ester. Unlike other reagents, DBCO is very hydrophobic, and its reaction requires the addition of organic solvents, such as DMSO (20-50 v %); moreover, the incorporation of DBCO into the polymer results in a significant shrink in hydrodynamic size (see FIG. 3e). To simplify the entire synthetic process, it was herein discovered that polymerization, azidation, and click reactions can be seamlessly conducted in one pot. Following purification through either tangential flow filtration or centrifugal dialysis, the polymer with a high degree of chain-end functionality can be easily obtained with an excellent yield of greater than 85% in only 6-8 hours.

The gelation of PCB-MAI at neutral pH occurred almost instantly upon the addition of crosslinkers such as dithiothreitol (DTT). To slow down the gelation and form a homogeneous gel, it was necessary to decrease the pH of the medium, a condition that is not ideal for hPSCs. On the other hand, the gelation of PCB-VS at neutral pH proceeded too slowly (>30 min) and required the elevation of pH, which is again not suitable for hPSCs. In comparison, PCB-DBCO (60 kDa, final concentration: 8% m/v) was found to be able to form gels with PEG₃-diazide in PBS (pH 7.4) at 37 °C within 5 minutes as determined by a vial inversion test. Rheology tests showed the time for complete gelation is dependent upon the polymer concentration but usually takes less than 30 minutes at 37 °C. When compared to work using difluorocyclooctyne (DIFO) as the chain-end functionality, not only is the rate of gelation of PCB-DBCO much faster, the degree of chain-end functionality can be directly determined by NMR, as shown in FIG. 3d, which makes it much easier to develop the formulation. The stiffness of the gels, as determined from compression tests, can be readily tuned by either changing the polymer concentration (6% to 14%) or changing the PCB molecular weight. With 60 kDa PCB-DBCO, the gel's Young's modulus can be fine-tuned within the range of 1-5 kPa, while the use of 30 kDa PCB-DBCO permits the stiffness of gel to be varied from 1 to 20 kPa (FIG. 3c), owing to the high degree of chain-end functionalization.

PCB Hydrogels are Biocompatible for hPSCs

An important criterion for developing a 3D culture system for hPSC expansion and differentiation is biocompatibility, or the ability of a material to be nontoxic to living cells. Initial attempts used PCB-MAI and PCB-VS hydrogels for iPSC expansion as they are easy and less costly to synthesize. However, high toxicities and low viability were encountered when encapsulating iPSCs in these gels, in addition to inadequate gelation times (1-2 seconds for PCB-MAI and 1-2 hours for PCB-VS). PCB-MAI was also compared to PEG-MAI hydrogels, and a comparable high cell death was found for both polymers with MAI chain-ends after only 24 hours, which suggests that this was due to an issue in the chain-end as opposed to the polymer backbone. This result indicated a need for bioorthogonal-based chemistries so as to avert reaction with critical cellular processes.

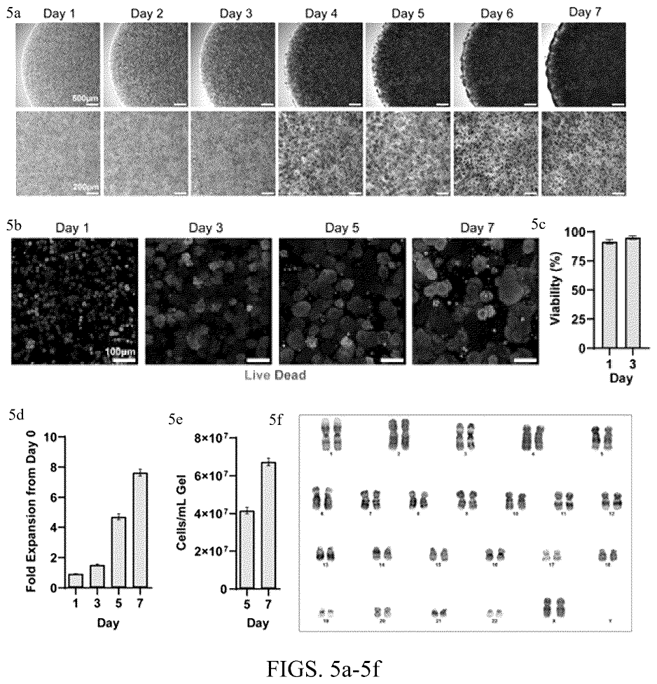

Further testing with azide-terminated PCB and PCB-DBCO gels showed not only viable cells after 1 day of exposure, but also cell growth. Based on preliminary viability evaluations of different chain-ends and modulus testing, an 8% PCB-60 kDa-DBCO hydrogel formulation (PAMAM core, method A; MMP-degradable EK(N₃)GPQGIWGQK(N₃)E as the crosslinker) for 3D hPSC culture was settled upon as this provided a Young's modulus of about 2 kPa which is consistent with the Young's modulus reported during embryogenesis. Cyclo(RGD) was further incorporated by copolymerization to permit cell attachment within the hydrogel. iPSCs were embedded in PCB hydrogels as described and plated as droplets in 24 and 6 well plates. These hydrogel droplets resulted in 3D iPSC spheroid growth over a week of culture (see FIG. 5a).

To determine the viability of the PCB-cultured iPSCs, live/dead staining was performed on days 1, 3, 5, and 7 of the culture (see FIG. 5b). The results indicated high cell viability with ~90% cells viable after 24 hours of encapsulation and ~95% viable at day 3 of culture, suggesting that both gelation with PCB-DBCO and cell release with collagenase are biocompatible (see FIG. 5c). Cell proliferation over time was also evaluated, and sustained cell growth from day 0 was observed (see FIG. 5d). PCB hydrogels generated 40 million cells per mL gel and 70 million cells per mL gel after 5 and 7 days of culture, respectively (see FIG. 5e). Moreover, karyotype analysis of hPSCs after a week of culture inside the PCB hydrogel was normal, which demonstrate that the PCB hydrogel does not induce chromosomal abnormalities in hPSCs despite achieving high cell proliferation (see FIG. 5f).

PCB Hydrogels Maintain and Promote hPSC Pluripotency

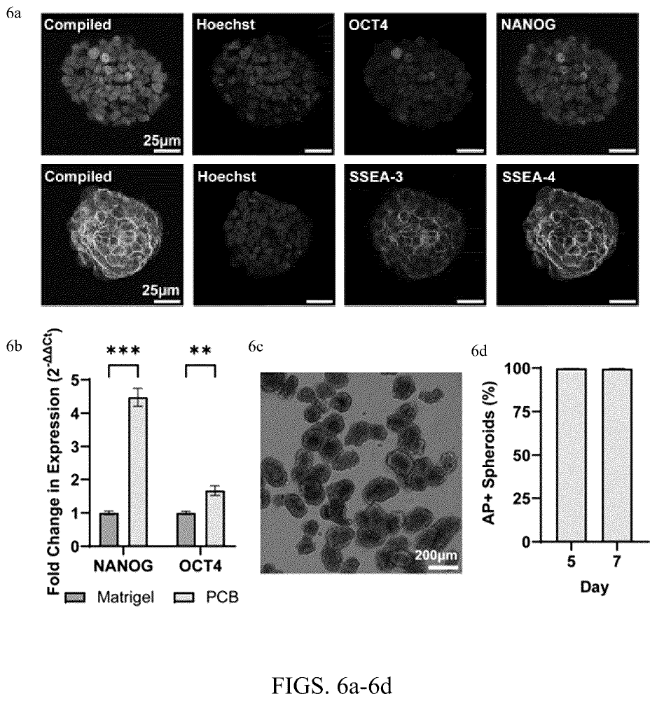

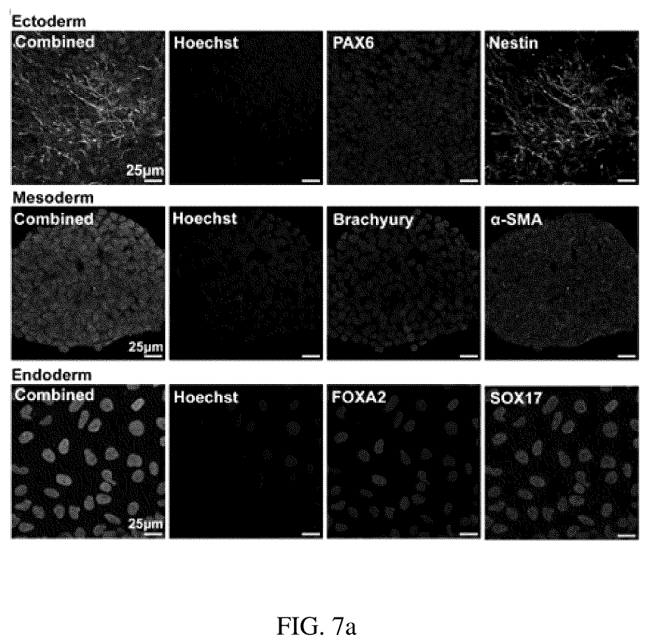

A key trait of hPSCs is their ability to maintain and regulate pluripotency. Pluripotency is often measured through the expression of pluripotency markers, such as OCT4, NANOG, SSEA-3, and SSEA-4. Both OCT4 and NANOG are transcription factors known to regulate hPSC self-renewal and stem cell fate. As well known, NANOG expression is required for preventing the differentiation of pluripotent stem cells while OCT4 is highly expressed in pluripotent cells and is lost upon differentiation. Similarly, as also well known, SSEA-3 and SSEA-4 are cell surface antigens expressed on ESCs during preimplantation development and quickly disappear by both iPSCs and ESCs during differentiation. The present immunofluorescence staining of pluripotent markers OCT4, NANOG, SSEA-3, and SSEA-4 demonstrate that hPSCs proliferated inside PCB hydrogels over a week and retain high pluripotency expression (see FIG. 6a). Furthermore, RT-qPCR analysis shows significantly higher expression of NANOG and OCT4 in hPSCs expanded inside PCB hydrogels compared to hPSCs expanded on Matrigel®, which suggests that the PCB hydrogel is promoting pluripotency (see FIG. 6b). Another measure for pluripotency is alkaline phosphatase (AP) activity. As well known, AP expression can be detected at the two-cell stage and is strictly expressed in the inner cell mass in the late stage of the blastocyst. Upon differentiation, AP expression is downregulated and lost making AP analysis a useful tool for determining hPSC pluripotency.

The above AP staining result further confirms high pluripotency in PCB-cultured hPSCs. Nearly all spheroids were observed to be AP+ after both 5 and 7 days of expansion inside PCB hydrogels (see FIG. 6c). AP quantifications confirmed that PCB-cultured hPSC spheroids were nearly 100% AP+ at day 5 and 7 post-encapsulation (see FIG. 6d). This suggests that not only is there high expression of pluripotency in iPSCs expanded in PCB hydrogels, but that the pluripotency expression is homogeneous throughout the cell population.

PCB Hydrogels Retain hPSC Differentiation Potential and Permit 3D Differentiation

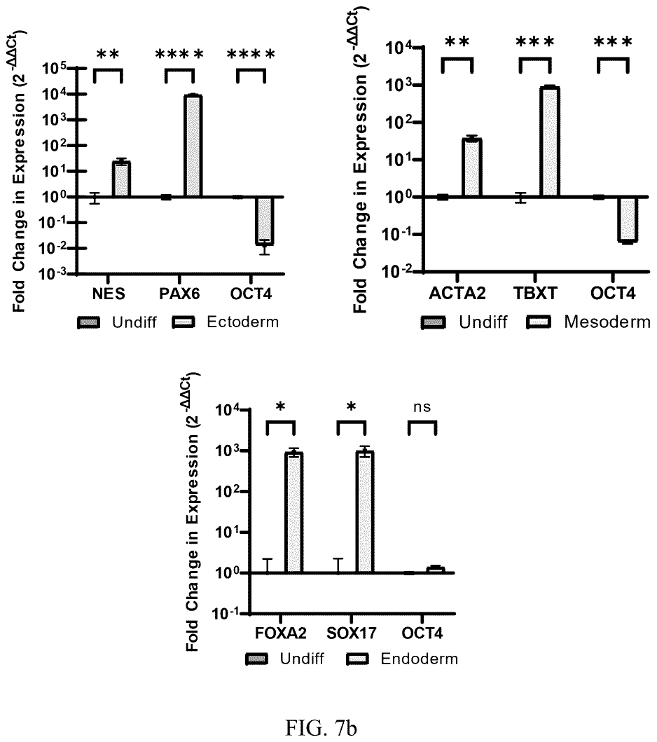

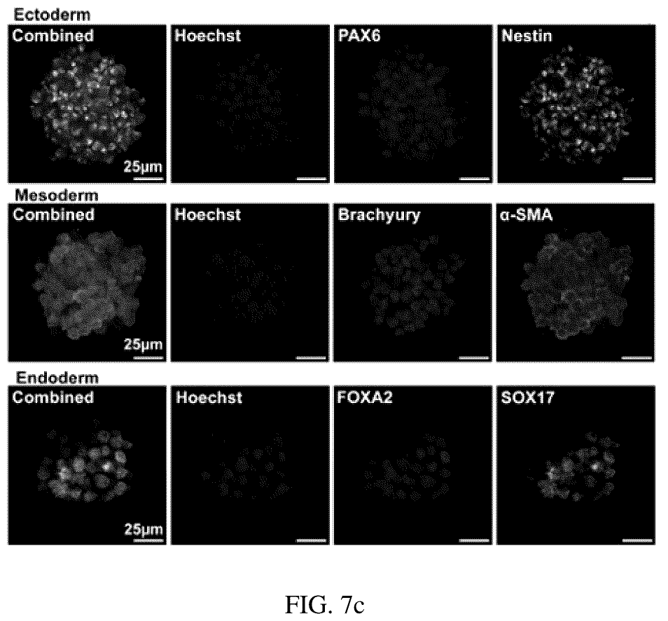

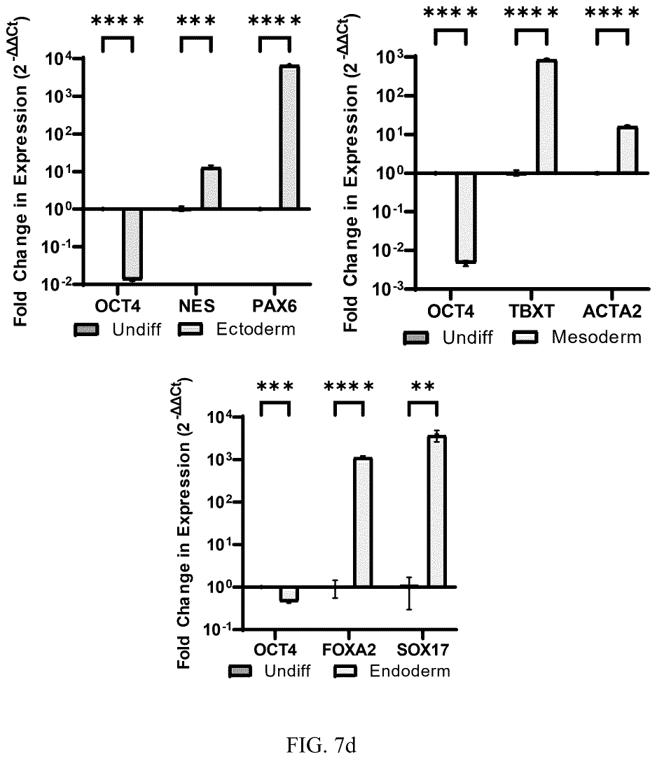

Another critical feature of hPSCs is their ability to undergo multi-lineage differentiations. To evaluate whether hPSCs expanded in the PCB hydrogel retain differentiation capabilities, iPSCs were first cultured in the PCB hydrogel over 5 days, and the cells were released from the hydrogel, the spheroids were dissociated to single cells for replating, and the cells were differentiated to the ectoderm, mesoderm, and endoderm germ layers using the commercial differentiation kit. Immunofluorescence staining and RT-qPCR were performed to detect the presence and quantity of germline specific markers. These markers included Nestin (NES) and PAX6 for ectoderm differentiated cells, Brachyury (TBXT) and α-SMA (ACTA2) for mesoderm differentiated cells, and FOXA2 and SOX17 for endoderm differentiated cells. OCT4 was also used as a negative control for RT-qPCR results as OCT4 is a marker for pluripotency, and its expression is expected to decrease upon hPSC differentiation. Based on immunofluorescence staining of ectoderm differentiated cells, a strong expression of nuclear PAX6 was observed in nearly every cell as well as expression of the cytoskeletal protein Nestin throughout the cells (see FIG. 7a). Both markers are indicative of neuron-related cells, which confirms differentiation to the ectoderm germ layer.

In mesoderm differentiated cells, strong expression was noted in Brachyury in the nucleus of nearly each cell and expression of α-SMA in the cytoplasm of nearly every cell (see FIG. 7a). As well known, Brachyury is a transcription factor required for mesoderm differentiation while α-SMA is a cytoskeletal protein commonly expressed by smooth muscle cells and myofibroblasts. Expression of these two markers in mesoderm differentiated hPSCs indicate that these PCB-cultured hPSCs successfully differentiated to the mesoderm germ layer. Moreover, in endoderm differentiated cells, expression of both FOXA2 and SOX17 was observed in the nucleus of each cell (see FIG. 7a). As also well known, FOXA2 is a critical transcription factor for the formation of the primitive foregut while SOX17 expression is required for definitive endoderm commitment. Expression of both FOXA2 and SOX17 in the nucleus of each cell confirms endoderm specification.

Similarly with RT-qPCR, expression of both NES and PAX6 were significantly higher in differentiated ectoderm samples compared to undifferentiated iPSCs with a nearly 25-fold higher expression of NES and a 9000-fold higher expression of PAX6 in the ectoderm differentiated samples (see FIG. 7b). Expression of OCT4 was also significantly decreased in the ectoderm differentiated samples by 75-fold, which further confirms cell differentiation. In the mesoderm differentiated samples, ACTA2 expression was significantly greater than undifferentiated hPSCs by 37-fold while TBXT expression was significantly greater by 900-fold (see FIG. 7b). Like ectoderm differentiated cells, expression of OCT4 was significantly decreased from undifferentiated hPSCs by 16-fold. In endoderm differentiated samples, the expression of FOXA2 and SOX17 was significantly greater than undifferentiated hPSCs by 930-fold and 1000-fold, respectively (see FIG. 7b). Taken together, these results demonstrate that hPSCs expanded in 3D PCB hydrogels retain trilineage differentiation capabilities, thereby making PCB hydrogels a valuable system for 3D hPSC culture. To generate large quantities of hPSCs for differentiations, the quality of the hPSCs is paramount. Reduced differentiation potential due to method of expansion would limit clinical applications and potentially patient outcomes. Using 3D PCB hydrogels for hPSC expansion provides a viable method for large-scale culture while maintaining necessary cell properties.

It was herein further investigated whether the PCB hydrogel could act as a platform to achieve 3D differentiation of hPSCs. To determine this, iPSCs were seeded in the PCB hydrogel with cyclo(RGD) as described, incubated with complete hPSC medium for 24 hours to allow for cell acclimation to the 3D environment, and then the cells were differentiated to the ectoderm, mesoderm, and endoderm germ layers using the commercial differentiation kit. Immunofluorescence staining and RT-qPCR markers included Nestin (NES) and PAX6 for ectoderm differentiated cells, Brachyury (TBXT) and α-SMA (ACTA2) for mesoderm differentiated cells, FOXA2 and SOX17 for endoderm differentiated cells, and OCT4 for pluripotent cells. Immunofluorescence staining revealed that the ectoderm generated cells had expression of PAX6 and Nestin in nearly all cells, confirming successful differentiation to the ectoderm lineage (FIG. 7c). Similarly, mesoderm differentiated cells had high expression of Brachyury and α-SMA demonstrating effective mesoderm commitment of iPSCs differentiated inside the PCB hydrogel (FIG. 7c). Finally, in endoderm differentiated cells, the nucleus of each cell strongly expresses FOXA2 and SOX17, which also confirms successful endoderm differentiation (FIG. 7c).

Quantifications of gene expression levels with RT-qPCR found that both NES and PAX6 were significantly higher in differentiated ectoderm samples compared to undifferentiated iPSCs by 13-fold and 6700-fold, respectively (see FIG. 7d). OCT4 was also significantly downregulated 76-fold in ectoderm differentiated samples. Mesoderm differentiated samples had significantly higher expression of TBXT at 850-fold and ACTA2 at 16-fold compared to undifferentiated iPSCs (see FIG. 7d). OCT4 was similarly downregulated in mesoderm differentiated cells inside the PCB hydrogel by 215-fold. Endoderm differentiated samples had significant upregulation of FOXA2 and SOX17 by 1100-fold and 3700-fold and downregulation of OCT4 (see FIG. 7d). These results highlight the potential of the PCB hydrogel to not only expand hPSCs while maintaining pluripotency and differentiation capabilities, but to also enable other applications like 3D differentiations. Compared to other scaffolds like PEG where hPSC differentiations were attempted inside the PEG hydrogel and PAX6 expression in ectoderm differentiated cells only achieved an 11-fold increase compared to undifferentiated iPSCs, it has herein been demonstrated that iPSCs differentiated in the PCB hydrogel achieve a soaring 6700-fold increase in PAX6 expression. Due to PCB's superhydrophilic nature, PCB creates a “clean background” in which specific cell-adhesive and cell-signaling moieties can easily be added, in which drug screening can be performed for organoids and spheroids, and in which cells can be in contact with microenvironments that are xenogenic-free, reproducible, and easy to scale-up.

### CONCLUSIONS

In this work, a highly efficient method was developed to access end-functionalized zwitterionic polymers. Using a one-pot aqueous Cu(0)-mediated LRP/azidation/click reaction, hydrolytically stable, end-functionalized poly(carboxybetaine acrylamide) (PCB) polymers of tunable molecular weight, narrow dispersity, and high end-group fidelity were synthesized with excellent yield. Water-soluble bioactive molecules can be efficiently incorporated into the polymer via direct copolymerization instead of post-polymerization modification, making the independent control of scaffold stiffness and bioactivity possible. PCB-DBCO can be readily crosslinked with bis(azide)s to form a hydrogel under physiological conditions with a favorable rate profile, and the stiffness of the gel can be tuned by simply adjusting the polymer concentration and/or PCB molecular weight. When an MMP-degradable peptide was used as a crosslinker, the gel is amenable to cell-mediated degradation and can be used for direct cell encapsulation. High viability, sustained cell growth, and normal karyotype over 7 days of culture supports the biocompatibility of the PCB hydrogel for hPSCs. High NANOG, OCT4, SSEA-3, SSEA-4, and AP expression in PCB-cultured hPSCs suggests that the PCB hydrogel preserves and promotes pluripotency. Additionally, successful germ layer differentiations demonstrate that hPSC expansion within PCB hydrogels does not inhibit hPSC differentiation capabilities and that the PCB hydrogel can be used for 3D hPSC differentiation and organoid generation. In view of the significantly improved synthetic accessibility, excellent biocompatibility, and exceptional performance in the culture of sensitive cells, this newly developed zwitterionic hydrogel system has been shown capable of replacing existing hydrogels as a next-generation synthetic scaffold for stem cell expansion, differentiation, and organoid growth.

While there have been shown and described what are at present considered the preferred embodiments of the invention, those skilled in the art may make various changes and modifications which remain within the scope of the invention defined by the appended claims.

1. A hydrogel composition comprising a star polymer core containing at least three arms, wherein each arm contains a copolymer comprising zwitterionic monomer units and peptide-functionalized monomer units, wherein the peptide functions as a cell adhesive ligand, and wherein the hydrogel composition contains a biodegradable or non-biodegradable linker.

2. The hydrogel composition of claim 1, comprising first (1^{st}) units and second (2^{nd}) units bonded to each other and having the following structure: 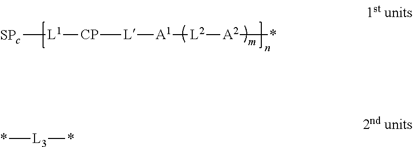
wherein:
SP_{c} is a star polymer core containing n arms, wherein n is at least 3;
L¹ and L′ are independently selected from a bond or a linker containing at least one carbon atom and optionally one or more heteroatoms selected from O, N, and S;
CP is a copolymer comprising zwitterionic monomer units and peptide-functionalized monomer units, wherein the peptide functions as a cell adhesive ligand;
A¹ is a reaction product between first and second reactive functional groups;
L², independently in each instance, is a linker containing at least one carbon atom and optionally one or more heteroatoms selected from O, N, and S;
A², independently in each instance, is a reaction product between first and second reactive functional groups;
m is 0, 1, 2, or 3; and
L³ is a biodegradable or non-biodegradable linker;
wherein the asterisks (*) in 1^{st} units represent connection points with asterisks in 2^{nd} units, and the hydrogel composition has a multiplicity of 1^{st} and 2^{nd} units.

3. The hydrogel composition of claim 2, wherein SP_{c} is a poly(amidoamine) (PAMAM) dendrimer, poly(propyleneimine) (PEI) dendrimer or poly(etherhydroxylamine) (PEHAM), wherein the dendrimer generation is 0-3.

4. The hydrogel composition of claim 2, wherein SP_{c} is a polyamidoamine (PAMAM) core.

5. The hydrogel composition according to claim 2, wherein n is at least 4.

6. The hydrogel composition according to claim 2, wherein m is 1.

7. The hydrogel composition according to claim 1, wherein the zwitterionic monomer units are carboxybetaine acrylamide monomer units or carboxybetaine methacrylate monomer units.

8. The hydrogel composition according to claim 1, wherein the peptide is selected from the group consisting of RGD, RGDS, c(RGDyK), IKVAV, YIGSR, PHSRN, and REDV, DDFVFYVGGYPS, TALKFYLQGPEP, LRLVSYSGVLFF, ASKAIQVFLLGG, LDGTGFARISFD, and QQNLGSVNVSTG.

9. The hydrogel composition according to claim 2, wherein L³ is a MMP-degradable peptide.

10. The hydrogel composition according to claim 2, wherein L³ is a non-biodegradable linker.

11. The hydrogel composition according to claim 2, wherein A¹ is a reaction product between an azide and a terminal alkyne; an azide and a cycloalkyne; a thiol and a maleimide; a thiol and a vinylsulfone; a thiol and a pyridyl disulfide; a thiol and an arylate; or an amine and a succinimidyl ester.

12. The hydrogel composition of claim 11, wherein A¹ is a reaction product between an azide and a cycloalkyne.

13. The hydrogel composition of claim 12, wherein the cycloalkyne is selected from the group consisting of azadibenzocyclooctyne (DBCO), bicyclo[6.1.0]nonyne (BCN), and difluorinated cyclooctyne (DIFO).

14. The hydrogel composition according to claim 2, wherein A² is a reaction product between an azide and a terminal alkyne; an azide and a cycloalkyne; a thiol and a maleimide; a thiol and a vinylsulfone; a thiol and a pyridyl disulfide; a thiol and an arylate; or an amine and a succinimidyl ester.

15. The hydrogel composition of claim 14, wherein A² is a reaction product between an azide and a cycloalkyne.

16. The hydrogel composition of claim 15, wherein the cycloalkyne is selected from the group consisting of azadibenzocyclooctyne (DBCO), bicyclo[6.1.0]nonyne (BCN), and difluorinated cyclooctyne (DIFO).

17. A method of forming the hydrogel composition of claim 1, the method comprising the following steps: 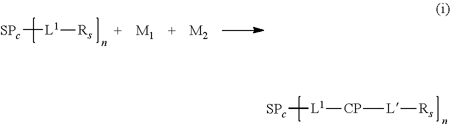
wherein:
SP_{c} is a star polymer (SP) core containing n arms, each arm containing a bond or linker (L¹) attached to a terminal reactive site (Rₛ) from which polymer growth can be initiated, wherein n is at least 3; wherein L¹ and L′ are independently selected from a bond or a linker containing at least one carbon atom and optionally one or more heteroatoms selected from O, N, and S;
M₁ is a zwitterionic monomer and M₂ is peptide-functionalized monomer, wherein the peptide functions as a cell adhesive ligand;
and
CP is a copolymer containing: (a) zwitterionic monomer units resulting from polymerization of M₁ and (b) peptide monomer units resulting from polymerization of M₂;
(ii) functionalizing SP_{c} [symbol] L¹-CP-L′-Rₛ]ₙ with a first reactive functional group R¹
to form the following structure:
SP_{c} [symbol] L¹-CP-L′-R¹]ₙ, wherein steps (i) and (ii) are optionally performed simultaneously; and
(iii) reacting SP_{c} [symbol] L¹-CP-L′-R¹]ₙ with R²-L³-R³ to form the hydrogel composition containing 1^{st} and 2^{nd} units, wherein:
R² and R³ are second and third reactive functional groups, respectively, and L³ is a biodegradable or non-biodegradable linker; wherein R¹ and R² react to form a reaction product A¹, and R¹ and R³ react to form another reaction product A¹, to result in the 1^{st} units and 2^{nd} units having the following structures: 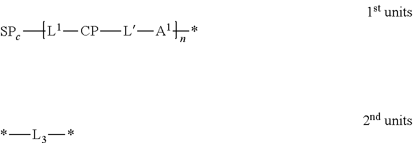
wherein the asterisks (*) in 1^{st} units represent connection points with asterisks in 2^{nd} units, and the hydrogel composition has a multiplicity of 1^{st} and 2^{nd} units.

18. The method of claim 17, wherein step (ii) further comprises an intermediate crosslinking step as follows:
reacting SP_{c} [symbol] L¹-CP-L′-R¹]ₙ with R^{1′}-L²-R^{2′} to form the following SP_{c} [symbol] L¹-CP-L′-A¹-L²-R^{2′}]ₙ,
wherein R¹, R^{1′}, and R^{2′} are reactive functional groups, and R¹ and R^{1′} react to form a reaction product A¹, and L² is a linker containing at least one carbon atom and optionally one or more heteroatoms selected from O, N, and S; and
reacting SP_{c} [symbol] L¹-CP-L′-A¹-L²-R^{2′}]ₙ with R²-L³-R³ to form the
hydrogel composition containing 1^{st} and 2^{nd} units, wherein:
R² and R³ are second and third reactive functional groups, respectively, and L³ is a biodegradable or non-biodegradable linker; wherein R^{2′} and R² react to form a reaction product A², and R^{2′} and R³ react to form another reaction product A², to result in the 1^{st} units and 2^{nd} units having the following structures: 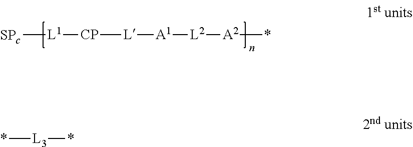
wherein the asterisks (*) in 1^{st} units represent connection points with asterisks in 2^{nd} units, and the hydrogel composition has a multiplicity of 1^{st} and 2^{nd} units.

19. The method according to claim 17, wherein SP_{c} is a poly(amidoamine) (PAMAM) dendrimer, poly(propyleneimine) (PEI) dendrimer or poly(etherhydroxylamine) (PEHAM), wherein the dendrimer generation is 0-3.

20. The method according to claim 17, wherein SPc is a polyamidoamine (PAMAM) core.

21. The method according to claim 17, wherein n is at least 4.

22. The method according to claim 17, wherein the zwitterionic monomer units are carboxybetaine acrylamide monomer units or carboxybetaine methacrylate monomer units.

23. The method according to claim 17, wherein the peptide is selected from the group consisting of RGD, RGDS, c(RGDyK), IKVAV, YIGSR, PHSRN, REDV, DDFVFYVGGYPS, TALKFYLQGPEP, LRLVSYSGVLFF, ASKAIQVFLLGG, LDGTGFARISFD, and QQNLGSVNVSTG.

24. The method according to claim 17, wherein L³ is a MMP-degradable peptide.

25. The method according to claim 17, wherein L³ is a non-biodegradable linker.

26. The method according to claim 17, wherein A¹ is a reaction product between an azide and a terminal alkyne; an azide and a cycloalkyne; a thiol and a maleimide; a thiol and a vinylsulfone; a thiol and a pyridyl disulfide; a thiol and an arylate; or an amine and a succinimidyl ester.

27. The method of claim 26, wherein A¹ is a reaction product between an azide and a cycloalkyne.

28. The method of claim 27, wherein the cycloalkyne is selected from the group consisting of azadibenzocyclooctyne (DBCO), bicyclo[6.1.0]nonyne (BCN), and difluorinated cyclooctyne (DIFO).

29. A method of culturing and containing cells comprising combining cells with the hydrogel composition according to claim 1 under conditions to promote cell survival, maintenance, cell product production, or cell growth.

30. The method of claim 29, wherein the cells are selected from the group consisting of stem cells, stem cell-derived cells and organoids, spheroids, cancer organoids, healthy organoids, diseased organoids, pancreatic islets, neutrophils, monocytes, T cells, and NK cells.

31. The method according to claim 29, wherein the method results in production of biological tissue, such as spheroids and organoids, and biological products derived from the cells, such as extracellular vesicles.

32. The method according to claim 29, wherein the cells are released from the hydrogel composition after culturing or containment.

33. The method of claim 32, wherein the released biological tissue is implanted into a living organism under conditions that are non-degradable or MMP-degradable.

34. The method of claim 29, wherein the hydrogel composition is implanted into a living organism, wherein the hydrogel composition may or may not contain cells when implanted, such as for the protection of beta cells for sustained insulin release (hydrogel containing cells) or for extended drug release from a hydrogel depot, for the creation of an empty scaffold in which native cells can invade and remodel, or for use as a functional tissue substitute such as for cartilage tissue replacement (cell free hydrogel).

35. The method according to claim 29, wherein continuous exposure of a Rho/ROCK inhibitor is not required for cell survival or growth.


SEQUENCE LISTING (15 sequences)

SEQ ID NO: 1 - protein, 4 aa, synthetic construct
  REGION 1..4: note = Peptide Sequence
RGDS

SEQ ID NO: 2 - protein, 5 aa, synthetic construct
  REGION 1..5: note = Peptide Sequence
  MOD_RES 4: note = Tyr is in D form
RGDYK

SEQ ID NO: 3 - protein, 5 aa, synthetic construct
  REGION 1..5: note = Peptide Sequence
IKVAV

SEQ ID NO: 4 - protein, 5 aa, synthetic construct
  REGION 1..5: note = Peptide Sequence
YIGSR

SEQ ID NO: 5 - protein, 5 aa, synthetic construct
  REGION 1..5: note = Peptide Sequence
PHSRN

SEQ ID NO: 6 - protein, 4 aa, synthetic construct
  REGION 1..4: note = Peptide Sequence
REDV

SEQ ID NO: 7 - protein, 12 aa, synthetic construct
  REGION 1..12: note = Peptide Sequence
DDFVFYVGGYPS

SEQ ID NO: 8 - protein, 12 aa, synthetic construct
  REGION 1..12: note = Peptide Sequence
TALKFYLQGPEP

SEQ ID NO: 9 - protein, 12 aa, synthetic construct
  REGION 1..12: note = Peptide Sequence
LRLVSYSGVLFF

SEQ ID NO: 10 - protein, 12 aa, synthetic construct
  REGION 1..12: note = Peptide Sequence
ASKAIQVFLLGG

SEQ ID NO: 11 - protein, 12 aa, synthetic construct
  REGION 1..12: note = Peptide Sequence
LDGTGFARISFD

SEQ ID NO: 12 - protein, 12 aa, synthetic construct
  REGION 1..12: note = Peptide Sequence
QQNLGSVNVSTG

SEQ ID NO: 13 - protein, 7 aa, synthetic construct
  REGION 1..7: note = Peptide Sequence
CGRGDSG

SEQ ID NO: 14 - protein, 16 aa, synthetic construct
  REGION 1..16: note = Peptide Sequence
GKKCGPQGIWGQCKKG

SEQ ID NO: 15 - protein, 12 aa, synthetic construct
  REGION 1..12: note = Peptide Sequence
  MOD_RES 2: note = lysine is functionalized with azide
  MOD_RES 11: note = lysine is functionalized with azide
EKGPQGIWGQKE